(19) United States
(12) Patent Application Publication
(10) Pub. No.: US 2011/0236405 A1
(43) Pub. Date: Sep. 29, 2011

(54) COAGULATION FACTOR MODULATION FOR CONTROLLING TRANSPLANT ORGAN SIZE

(75) Inventors: Yair Reisner, Old Jaffa (IL); Anna Aronovich, Rehovot (IL); Dalit Tchorsh-Yutsis, Rehovot (IL); Gideon Rechavi, Tel-Aviv (IL)

(73) Assignee: Tel HaShomer Medical Research Infrastructure and Services Ltd., Ramat-Gam (IL)

(21) Appl. No.: 13/056,665

(22) PCT Filed: Jul. 19, 2009
(86) PCT No.: PCT/IL09/00707

§ 371 Date: Apr. 12, 2011

Related U.S. Application Data
(60) Provisional application No. 61/129,919, filed on Jul. 29, 2008.
(60) Provisional application No. 61/193,135, filed on Oct. 30, 2008.

(51) Int. Cl. A61K 39/00; A61K 38/36; A61P 7/02; A61K 38/37; A61K 38/48; A61P 41/00

(52) U.S. Cl. 424/184.1; 514/13.7; 514/14.1; 424/94.64

(57) ABSTRACT

A method of modulating transplant organ size in a subject in need thereof is disclosed. The method comprising: (a) administering to the subject an agent capable of modulating an activity or expression of a coagulation factor or an effector thereof; and (b) transplanting the organ into the subject; thereby modulating the transplant organ size in the subject.

### FIELD AND BACKGROUND OF THE INVENTION

The present invention, in some embodiments thereof, relates to coagulation factors and effectors of same, and more particularly, but not exclusively, to the modulation of same for control of transplant organ size.

Organ transplants are commonly used for treatment of organ failure, however, there is a major shortage in donor organs and the difference between supply and demand continues to grow every year. Transplantation of organs including kidney, heart, liver, lung and pancreas, are carried out following assessment of several factors including organ size, blood type, tissue type, medical urgency of the subject's illness and time already spent on the waiting list. The organ is offered first to the candidate who is the best match in all the above criteria, but nevertheless, recipients usually wait for prolonged periods of time before a transplant can be found and often no matched transplants are found resulting in the death of many patients every year.

Despite great progress in molecular genetics and embryogenesis, size control of tissues and organs remains a mystery. Organ size control during embryonic development or in tissue regeneration, involves a fine balance between cell growth, proliferation and death, maintained by extrinsic and intrinsic factors. Even though the size of an organ or organism depends largely on cell numbers and cell size, studies have found that the simple deregulation of cell proliferation or cell growth does not necessarily lead to changes in organ size. Some important insights into organ size control were provided in the past few decades from studies of the Drosophila imaginal disc model. In general, it seems that extrinsic mechanisms are associated with nutrition or systemic growth factors operating through the Insulin/PI3K and the TOR pathways, while intrinsic mechanisms are likely linked to patterning morphogenes and apoptosis-signaling complexes. Overall, experimental data suggest that organ size might be regulated by a ‘total mass checkpoint’ mechanism which functions to link the regulation of cell size and cell proliferation [Christopher et al., Curr Opin in Genet Develop (2001) 11(3) 279-286].

Recent evidence suggests that in some organs, such as the pancreas, organ size is intrinsically defined by the size of the stem cell pool committed to the development of the organ. Thus, using genetic manipulations leading to reduction of the size of the stem cell pool it was demonstrated that a smaller pancreas was generated in animals with reduced stem cells [Stanger et al., Nature (2007) 445: 886-91], while in contrast, similar genetic manipulations of liver stem cells did not affect the ability of the liver to regenerate and regain its normal size [Stanger et al., supra]. This difference might indicate that autonomous growth of the embryonic pancreas tissue exhibits total dependence on intrinsic elements, namely the size of the stem cell pool, in contrast to the embryonic liver, which is likely controlled by additional extrinsic factors. These recent observations are reminiscent of the early studies of Metcalf who showed that following transplantation of several pieces of embryonic thymus, each one attained the full size of an adult thymus [Metcalf D., Aust J Exp Biol Med Sci (1963) 41, SUPPL437-47], while transplanting embryonic spleen tissue, the combined size of all the spleen implants was similar in size to a single adult spleen [Metcalf D., Transplantation (1964) 2: 387-92].

As stated above, it is generally accepted that liver regeneration is not dependent on progenitor cells or stem cells but rather on extrinsic factors. According to the teachings of Michalopoulos and DeFrances [Michalopoulos and DeFrances, Science (1997) 276: 60-66] transplanting livers from large dogs into small dogs results in a gradual decrease in liver size until the size of the organ becomes proportional to the new body size. On the contrary, when baboon livers are transplanted into humans, the transplanted intact livers of baboon origin rapidly grow in size (within a week) until reaching the size of a human liver. These results demonstrate that liver mass is regulated and that signals from the body can have negative as well as positive effects on liver mass until the correct size is reached. Furthermore, according to their teachings, liver regeneration is an orchestrated response induced by specific external stimuli and involving sequential changes in gene expression, growth factor production, and morphologic structure. Many growth factors and cytokines, most notably hepatocyte growth factor, epidermal growth factor, transforming growth factor-α, interleukin-6, tumor necrosis factor-α, insulin and norepinephrine, appear to play an important role in liver regeneration.

### SUMMARY OF THE INVENTION

According to an aspect of some embodiments of the present invention there is provided a method of modulating transplant organ size in a subject in need thereof, the method comprising: (a) administering to the subject an agent capable of modulating an activity or expression of a coagulation factor or an effector thereof; and (b) transplanting the organ into the subject; thereby modulating the transplant organ size in the subject.

According to an aspect of some embodiments of the present invention there is provided a use of an agent capable of down-regulating an activity or expression of a coagulation factor or an effector thereof for enhancing a transplant organ size in a subject.

According to an aspect of some embodiments of the present invention there is provided a use of an agent capable of up-regulating an activity or expression of a coagulation factor or an effector thereof for decreasing a transplant organ size in a subject.

According to an aspect of some embodiments of the present invention there is provided a pharmaceutical composition comprising an agent capable of down-regulating an activity or expression of a coagulation factor or an effector thereof for enhancing a transplant organ size in a subject.

According to an aspect of some embodiments of the present invention there is provided a pharmaceutical composition comprising an agent capable of up-regulating an activity or expression of a coagulation factor or an effector thereof for decreasing a transplant organ size in a subject.

According to an aspect of some embodiments of the present invention there is provided an article of manufacture comprising a packaging material packaging an immunosuppressing agent and an agent capable of modulating an activity or expression of a coagulation factor or an effector thereof.

According to some embodiments of the invention, the modulating transplant organ size comprises enhancing the transplant organ size.

According to some embodiments of the invention, the agent is capable of down-regulating the activity or expression of the coagulation factor or an effector thereof.

According to some embodiments of the invention, the coagulation factor or an effector thereof is selected from the group consisting of Factor VIII, Factor X, Factor Xa, Prothrombin, Thrombin, Factor XIII, Factor XIIIa and PAR.

According to some embodiments of the invention, the agent is capable of down-regulating an activity or expression of Factor VIII in the subject.

According to some embodiments of the invention, the agent is capable of down-regulating an activity or expression of Factor Xa in the subject.

According to some embodiments of the invention, the agent is Clexane.

According to some embodiments of the invention, the agent is capable of down-regulating an activity or expression of Thrombin in the subject.

According to some embodiments of the invention, the agent is selected from the group consisting of Clexane and Dabigatran.

According to some embodiments of the invention, the agent is capable of up-regulating an activity or expression of antithrombin in the subject.

According to some embodiments of the invention, the agent is capable of down-regulating an activity or expression of PAR1 in the subject.

According to some embodiments of the invention, the agent is as set forth in SEQ ID NO: 15.

According to some embodiments of the invention, the agent is capable of down-regulating an activity or expression of PAR4 in the subject.

According to some embodiments of the invention, the agent is as set forth in SEQ ID NO: 16.

According to some embodiments of the invention, the agent further comprises G-CSF.

According to some embodiments of the invention, the agent is an oligonucleotide silencing agent.

According to some embodiments of the invention, the modulating transplant organ size comprises decreasing the organ size.

According to some embodiments of the invention, the agent is capable of up-regulating the activity or expression of the coagulation factor or an effector thereof.

According to some embodiments of the invention, the coagulation factor or an effector thereof is selected from the group consisting of Factor VIII, Factor X, Factor Xa, Prothrombin, Thrombin, Factor XIII, Factor XIIIa and PAR.

According to some embodiments of the invention, the agent is capable of up-regulating an activity or expression of Factor VIII in the subject.

According to some embodiments of the invention, the agent is capable of up-regulating an activity or expression of Thrombin in the subject.

According to some embodiments of the invention, the agent is capable of down-regulating an activity or expression of antithrombin in the subject.

According to some embodiments of the invention, the agent is selected from the group consisting of human Factor VIII, recombinant Factor VIII, porcine factor VIII, Factor X, Factor Xa, Prothrombin, Thrombin, activated prothrombin complex, desmopressin (DDAVP), Factor XIII and Factor XIIIa.

According to some embodiments of the invention, the organ comprises a solid tissue.

According to some embodiments of the invention, the organ comprises a liver.

According to some embodiments of the invention, the organ comprises a spleen.

According to some embodiments of the invention, the organ comprises a pancreas.

According to some embodiments of the invention, the organ is derived from a prenatal organism.

According to some embodiments of the invention, the organ is derived from a post natal organism.

According to some embodiments of the invention, the organ is derived from an adult.

According to some embodiments of the invention, the organ is derived from a xenogeneic donor.

According to some embodiments of the invention, the xenogeneic donor is a pig.

According to some embodiments of the invention, the organ is derived from an allogeneic donor.

According to some embodiments of the invention, the organ is derived from a syngeneic donor.

According to some embodiments of the invention, the organ is derived from a cadaver donor.

According to some embodiments of the invention, the subject is a human being.

According to some embodiments of the invention, the subject in need thereof has a hepatic disorder.

According to some embodiments of the invention, the subject in need thereof has a renal disorder.

According to some embodiments of the invention, the subject in need thereof has a pancreatic disorder.

According to some embodiments of the invention, the modulating an activity or expression of a coagulation factor or an effector thereof is effected prior to, concomitantly with or following transplantation.

According to some embodiments of the invention, the method further comprising conditioning the subject prior to transplanting so as to prevent organ rejection.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G depict transplantation of prenatal pig spleen tissues in NOD-SCID and factor VIII KO SCID mice. FIG. 1A depicts macroscopic view of an E-42 graft transplanted into NOD-SCID recipient mouse 3 months post transplantation; FIG. 1B depicts macroscopic view of an E-42 graft transplanted into factor VIII KO SCID recipient mouse 3 months post transplantation; FIG. 1C depicts the average weight of E42 pig spleen grafts in NOD-SCID versus factor VIII KO SCID recipients (n=20); FIGS. 1D-G depict analogous development as shown by H&E staining of E42 pig spleen grafts transplanted under the kidney capsule of a NOD-SCID mouse (FIG. 1D) and a Factor VIII KO-SCID mouse (FIG. 1E) and by endothelial pattern as illustrated with anti-pig CD31 (FIG. 1F) and (FIG. 1G), respectively.

FIGS. 2A-D depict enhancement of pig pancreas size and function transplanted in factor VIII KO-SCID mice. FIG. 2A depicts pig insulin serum levels following implantation of E42 pig pancreas into Factor VIII KO SCID and NOD-SCID mice. Data represents 4 independent experiments (P<0.005); FIG. 2B depicts morphometric analysis of growth of E42 pig pancreas implants. Both recipients were evaluated for graft volume (left column), beta cell volume (middle column) and for proportion of beta cells versus total volume (right column) 3 months after transplantation (n=5); FIG. 2C depicts normal histological findings following an E42 pig pancreas transplantation under the kidney capsule of a NOD-SCID mouse; and FIG. 2D depicts normal histological findings following an E42 pig pancreas transplantation under the kidney capsule of a Factor VIII KO-SCID mouse. Of note, pig insulin is marked by red while glucagon is represented by green.

FIGS. 3A-G depict enhancement of pig liver size and function transplanted in factor VIII KO-SCID mice. FIG. 3A depicts pig albumin serum levels following implantation of E42 pig liver precursor tissues into Factor VIII KO SCID and NOD-SCID mice. Data represents 3 independent experiments (P=0.032); FIGS. 3B-G depict increased growth and retained functionality of the liver grafts in Factor FIII KO-SCID recipient. FIGS. 3B-C depict H&E staining of the liver grafts from NOD-SCID and Factor VIII KO-SCID mice, respectively; FIGS. 3D-E depict immunohistological staining of pig albumin in NOD-SCID and Factor VIII KO-SCID mice, respectively; and FIGS. 3F-G depict periodic acid/Schiff (PAS) of the liver grafts from NOD-SCID and Factor VIII KO-SCID mice, respectively. Of note, the figures illustrate enhancement of growth and functionality of the liver grafts.

FIGS. 3H-I depict enhancement of pig spleen implant size in RAG^{-/-} FVIII KO mice. FIG. 3H depicts RAG^{-/-} mice and FIG. 3I depicts RAG^{-/-} hemophilic mice. Both figures show H&E staining of E42 pig spleen implants twelve weeks post transplantation. Bar stands for 2 mm.

FIGS. 4A-C depict potential checkpoints for excessive organ growth following transplantation of mouse and porcine fetal precursor tissues into SCID mice. FIG. 4A depicts transplantation of mouse embryonic spleen (E15), liver (E16) and pancreas (E16) under the kidney capsule of NOD-SCID or Factor VIII KO SCID mice. Of note, no difference in organ size was detected 3 months post transplantation; FIG. 4B depicts transplantation of porcine embryonic spleen (E42), pancreas (E42) and liver (E28) under the kidney capsule of NOD-SCID or Factor VIII KO SCID mice. Of note, as the original size of porcine organs is larger in comparison to mouse organs, it is assumed and illustrated that the stem cell pool in each embryonic porcine organ is initially greater than that of the mouse organ. This difference is reflected by the larger size of pig implant in NOD-SCID mice compared the growth displayed by embryonic mouse counterparts. However, marked overgrowth was detected upon transplantation of embryonic pig tissues into Factor VIII KO SCID mice compared to their growth in non-hemophilic NOD-SCID mice; FIG. 4C depicts gene expression analysis comparing embryonic pig spleen implants (E42) grown in non-hemophilic or Factor VIII KO recipients. Of note, the figure suggests that overgrowth control by Factor VIII may be mediated by modulation of graft intrinsic genes coding for proteins involved in growth, coagulation, differentiation and patterning.

FIGS. 5A-B depict enhanced splenomegaly induced by G-CSF in SCID Factor VIII KO mice compared to non-hemophilic NOD-SCID mice. FIG. 5A depicts a macroscopic view of splenomegaly in G-CSF treated and untreated mice; FIG. 5B depicts spleen weights in G-CSF treated and untreated mice or exogenous Hu Factor VIII infused SCID FVIII KO mice. Data represents 4 independent experiments (**P<0.0001; *P=0.033).

FIG. 6 depicts G-CSF induced splenomegaly in C57BL mice compared to C57BL hemophilic (C57BL Hem F8) mice. Spleen weights in mg are shown in the presence or absence of G-CSF.

FIG. 7 depicts a schematic representation of the coagulation cascade. Of note, Factor Xa is activated by Factor VIII and, in turn, Thrombin is activated by Factor Xa.

FIGS. 8A-C depict the effect of Clexane on G-CSF splenomegaly and on albumin secretion following embryonic pig transplantation in mouse models. FIG. 8A depicts a schematic representation of Clexane inhibition in the coagulation cascade; FIG. 8B depicts G-CSF induced splenomegaly in C57BL mice with and without administration of Clexane; FIG. 8C depicts enhancement of embryonic pig liver growth by Clexane administration in Rag^{-/-} mice. Pig albumin serum levels were detected by specific ELISA at 7, 14 and 21 days post implantation of E42 pig liver precursor tissue, in the presence or absence of Clexane administration. The results are compared to those obtained in hemophilic Rag^{-/-} factor VIII KO mice.

FIG. 9A depicts G-CSF induced splenomegaly in C57BL mice with and without Dabigatran administration. Spleen weights in mg are shown in the presence or absence of G-CSF and Dabigatran treatment.

FIG. 9B depicts enhancement of embryonic pig liver growth by Dabigatran administration in Rag−/− mice. Pig albumin serum levels, detected by specific ELISA, are shown at 7, 14 and 21 days after implantation of E42 pig liver precursor tissue in the presence or absence of Dabigatran administration. The results are compared to those obtained in hemophilic Rag−/− Factor VIII KO mice.

FIG. 10 depicts G-CSF induced splenomegaly in C57BL mice treated with PAR1 and PAR4 antagonists compared to C57BL mice. Spleen weights in mg are shown in the presence or absence of G-CSF and antagonist treatment.

FIG. 11 is a schematic illustration depicting overgrowth stimulus regulation by factors in the coagulation cascade.

### DESCRIPTION OF SPECIFIC EMBODIMENTS OF THE INVENTION

The present invention, in some embodiments thereof, relates to coagulation factors and effectors of same, and more particularly, but not exclusively, to the modulation of same for control of transplant organ size.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have surprisingly uncovered that various factors in the blood coagulation pathway play a key role in modulating transplant organ size without compromising tissue functionality. Apparently, but without being bound by theory, coagulation factors or absence thereof mediates this novel activity by modulating key genes known to participate in coagulation, endothelium formation, patterning and differentiation.

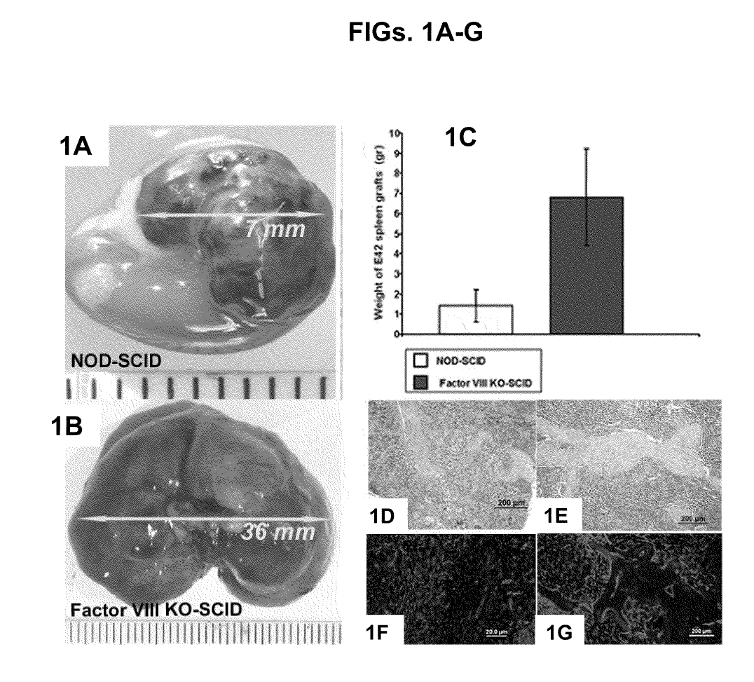

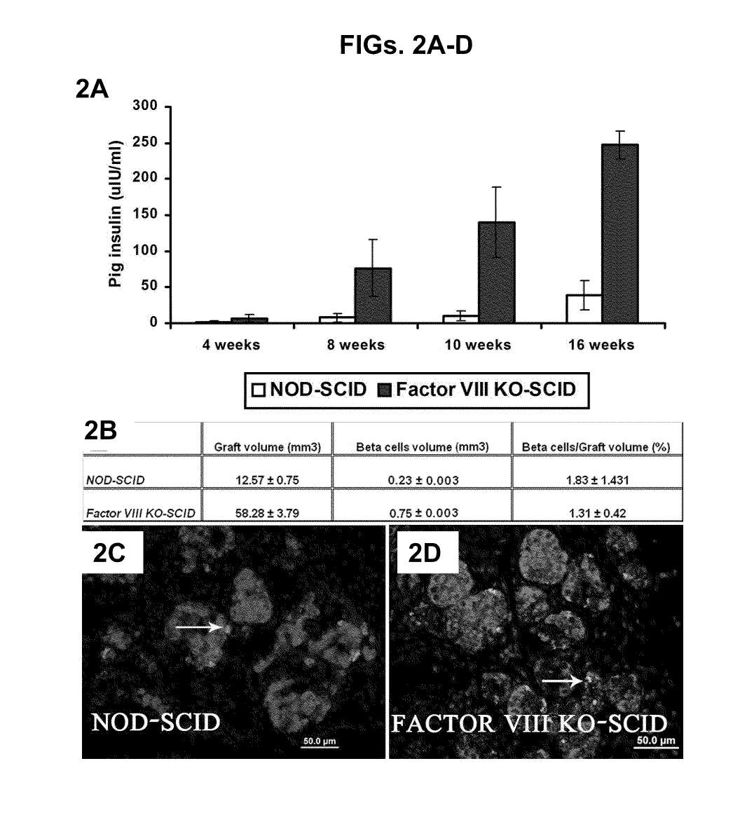

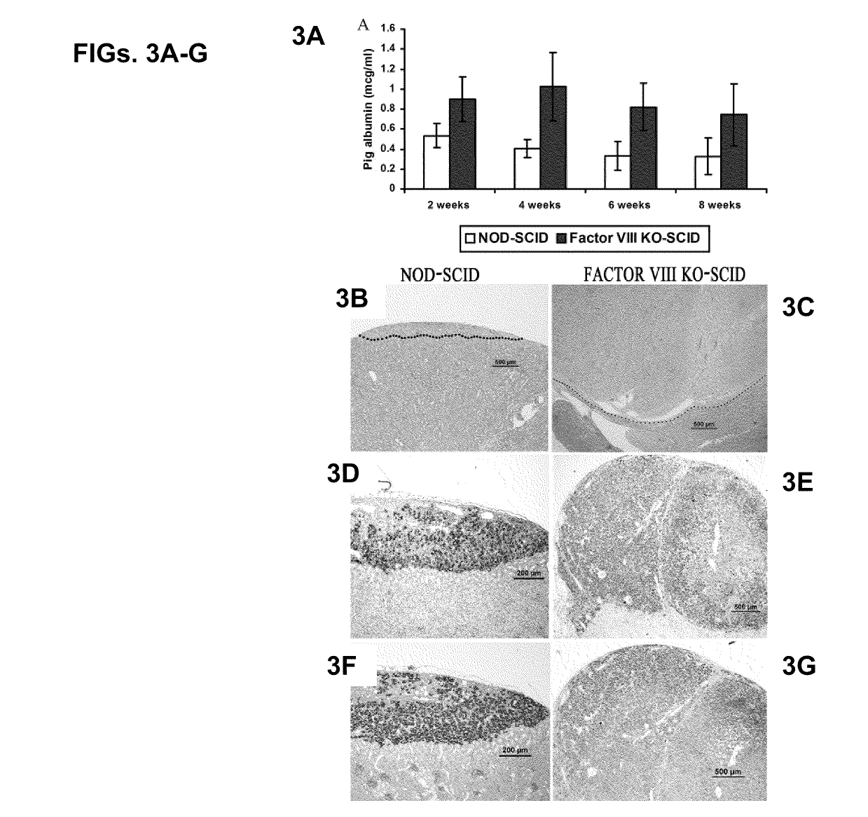

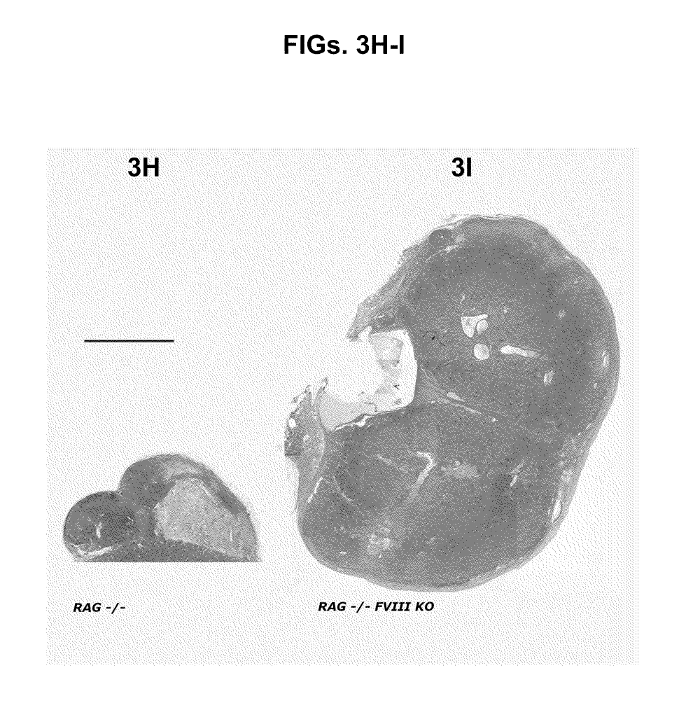

As is shown hereinbelow and in the Examples section which follows, the present inventors have shown that transplanted pig embryonic tissues grew to a larger size in hemophilic (Factor VIII KO) recipient mice in comparison to wild type mice. As is shown in FIGS. 1A-G, pig embryonic spleen implants depicted normal growth, development and vascularization patterns in Factor VIII KO mice while concomitantly displaying enhanced organ size (by a factor 2.76, 3 months post transplant). Similar results were obtained for transplantation of pig embryonic pancreatic and liver tissues. Specifically, the growth of transplanted pig pancreatic tissues in Factor VIII KO mice resulted in increased blood levels of pig insulin (FIG. 2A) correlating with the enhanced growth of pancreas size in these mice (by a factor 3, FIG. 2B). Likewise, transplantation of pig liver tissues into Factor VIII KO mice lead to a significant enhancement in implant size (by at least a factor of 2) and enhanced levels of pig albumin blood levels (FIG. 3A). The growing pig liver (FIGS. 3B-E) and pancreas (FIGS. 2C-D) exhibited similar architecture in both Factor VIII KO and wild-type recipients, thus despite the large organ size, functionality was maintained.

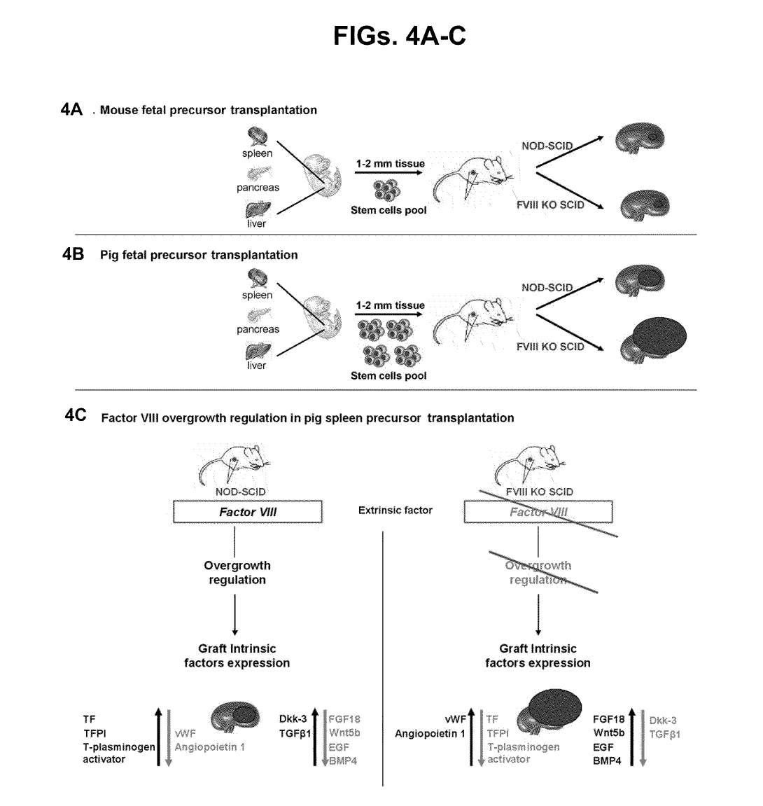

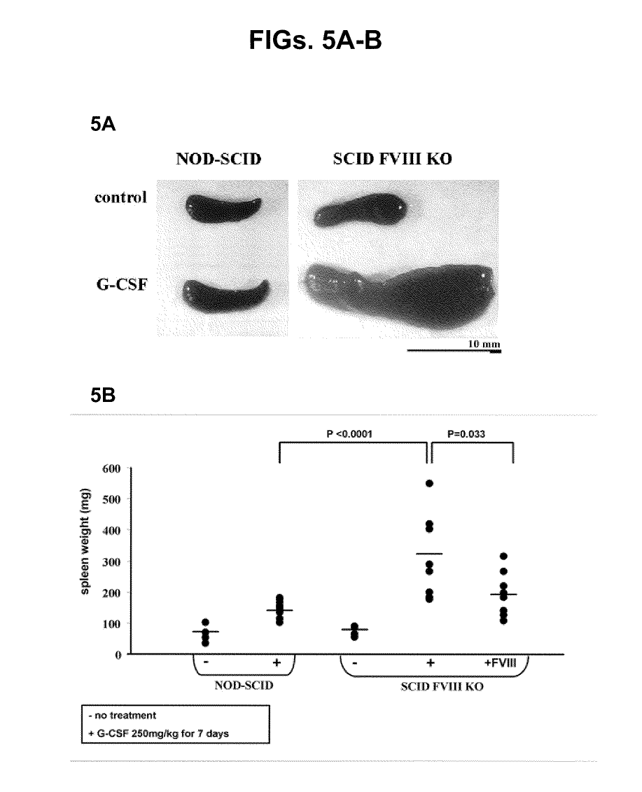

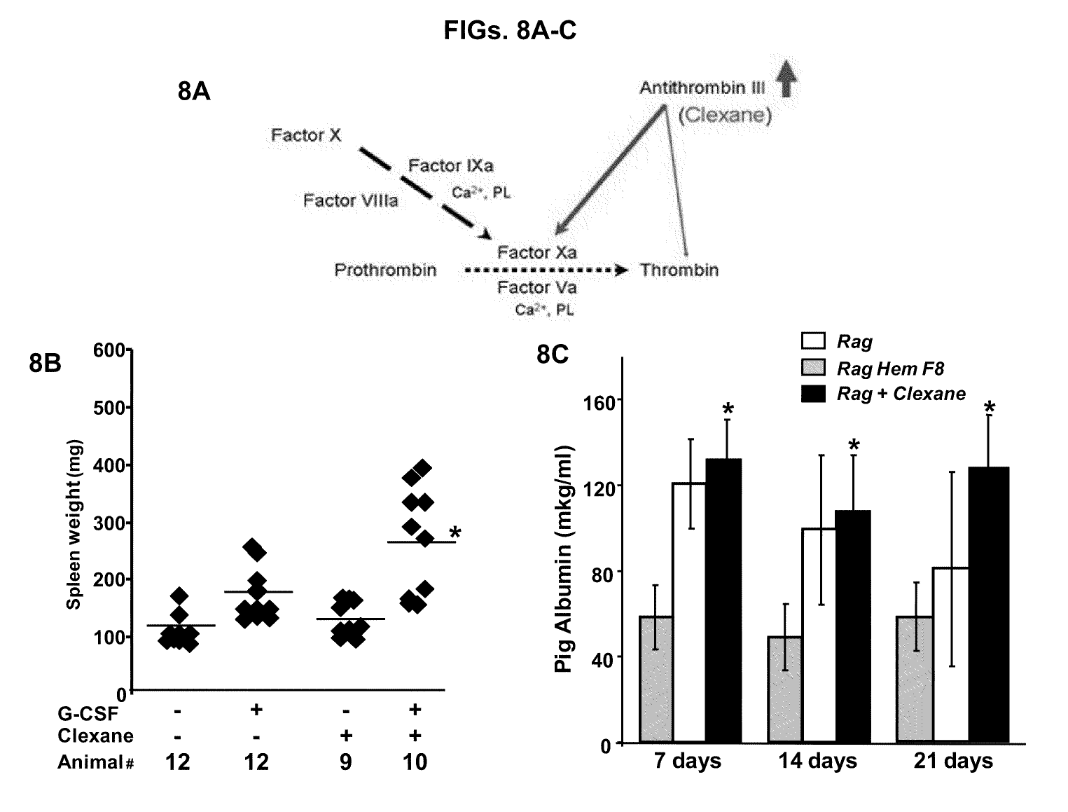

Moreover, it is shown herein that Factor VIII is involved in regulation of organ size in situations in which there is a drive for oversized growth. For example, treatment of hemophilic mice with G-CSF lead to splenomegaly in these mice (FIG. 5A-B). As depicted in FIGS. 4A-B, embryonic implants of different sources (e.g. pig, mouse) are endowed with stem cell pools of different sizes prior to transplantation. These tissues are therefore likely to exhibit different organ size upon completion of growth and differentiation. As a result of Factor VIII depletion, the hemophilic mice lacking the potential inhibitory activity (i.e. overgrowth checkpoint) exhibit much larger pig transplanted organ sizes (FIG. 4C). Similarly, down regulation of other coagulant factors, such as Factor Xa and Thrombin, had major influence on oversized organs (FIGS. 8B-C and 9). Taken together, all these findings substantiate the use of coagulation factors or effectors thereof for the modulation of transplant organ size according to the source of the organ and the intended use.

Thus, according to one aspect of the present invention there is provided a method of modulating transplant organ size in a subject in need thereof. The method comprising: administering to the subject an agent capable of modulating an activity or expression of a coagulation factor or an effector thereof; and transplanting the organ into the subject; thereby modulating the transplant organ size in the subject.

As used herein, the term “modulating” refers to a change in size of the transplanted organ in the host, either an increase (e.g., at least 5%, 10%, 15%, 20%, 30%, 50%, 100%, 200%, 250%, 400% or more) or a decrease (e.g., at least 5%, 10%, 15%, 20%, 30%, 50%, 100%, 200%, 250%, 400% or more). Modulation is typically determined with respect to an untreated subject (i.e., who was not subject to modulation of a coagulation factor or an effector thereof). Modulation can be determined by any method known to one of ordinary skill in the art, as for example by activity assays such as measurement of blood insulin or albumin levels for determination of pancreas or liver transplant organ sizes, respectively, or by using any suitable, widely practiced, imaging methods including computerized tomography (CT) and ultrasound imaging. If a plurality of observations are made, one skilled in the art can apply any routine statistical analysis to identify such modulations. Typically, according to some embodiments of the present invention, modulating transplant organ size is not accompanied by changes in functionality of the transplanted tissue.

As used herein, the phrase “subject in need thereof” refers to a mammal, preferably a human being, male or female at any age that is in need of organ transplantation. Typically the subject is in need of organ transplantation (also referred to herein as recipient) due to a disorder or a pathological or undesired condition, state, or syndrome, or a physical, morphological or physiological abnormality which is amenable to treatment via organ transplantation. Examples of such disorders are provided further below. Moreover, the subject is typically not diagnosed with a coagulation factor disorder.

As used herein, the term “organ” refers to a bodily tissue which may be transplanted in full or in part, including solid tissues and soft tissues. Exemplary organs which may be transplanted according to the present teachings include, but are not limited to, liver, pancreas, spleen, kidney, heart, lung, skin, intestine and lymphoid/hematopoietic tissues (e.g. lymph node, Peyer's patches thymus or bone marrow). It will be appreciated that the organ of the present invention is not an embryo or fetus.

As used herein, the phrase “transplant organ size” refers to the size of an organ transplanted from one body to another. The transplant organ size may be evaluated in comparison to the average size of an identical organ transplanted to a host of the same species, age group, medical condition and gender as the subject. Transplant organ size is evaluated post transplantation and optionally prior to the transplantation.

Transplanting the organ may be effected in numerous ways, depending on various parameters, such as, for example, the graft type; the type, stage or severity of the recipient's organ failure; the physical or physiological parameters specific to the subject; and/or the desired therapeutic outcome. Depending on the application and purpose, transplanting the organ may be effected using an organ originating from any of various mammalian species, by implanting the organ into various anatomical locations of the subject, using an organ consisting of a whole or partial organ or tissue, and/or by using a transplant consisting of various numbers of discrete organs, tissues, and/or portions thereof.

Optionally, when transplanting an organ of the present invention into a subject having a defective organ, it may be advantageous to first at least partially remove the failed organ from the subject so as to enable optimal development of the transplant, and structural/functional integration thereof with the anatomy/physiology of the subject.

Depending on the application, the method may be effected using an organ which is syngeneic or non-syngeneic with the subject.

As used herein, an organ which is “syngeneic” with the subject refers to an organ which is derived from an individual who is essentially genetically identical with the subject. Typically, essentially fully inbred mammals, mammalian clones, or homozygotic twin mammals are syngeneic.

Examples of syngeneic organs include an organ derived from the subject (also referred to in the art as an “autologous organ”), a clone of the subject, or a homozygotic twin of the subject.

As used herein, an organ which is “non-syngeneic” with the subject refers to an organ which is derived from an individual who is allogeneic or xenogeneic with the subject's lymphocytes.

As used herein, an organ which is “allogeneic” with the subject refers to an organ which is derived from a donor who is of the same species as the subject, but which is substantially non-clonal with the subject. Typically, outbred, non-zygotic twin mammals of the same species are allogeneic with each other.

As used herein, an organ which is “xenogeneic” with the subject refers to an organ which substantially expresses antigens of a different species relative to the species of a substantial proportion of the lymphocytes of the subject. Typically, outbred mammals of different species are xenogeneic with each other.

As is described and illustrated in the Examples section below, porcine organs were transplanted into immunodeficient mice which were hemophilic or non-hemophilic. These organs developed into well developed and tolerated functional organs of porcine lineage.

Porcine organs are widely considered to be a potentially ideal animal alternative to human organs for therapeutic transplantation in humans due to their morphological compatibility with the human anatomy, and due to their essentially unlimited supply which would overcome the restricted availability impediment inherent to prior art human organs [Auchincloss, H. and Sachs, D. H., Annu. Rev. Immunol. (1998) 16, 433-470; Hammerman, M. R., Curr. Opin. Nephrol. Hypertens. (2002) 11, 11-16].

Organs of porcine origin are preferably obtained from a source which is known to be free of porcine zoonoses, such as porcine endogenous retroviruses. Similarly, human-derived organs are preferably obtained from substantially pathogen-free sources.

Depending on the application and available sources, the organ may be obtained from a prenatal organism, postnatal organism, an adult or a cadaver donor.

An organ derived from a prenatal organism may be obtained from a fetus at any gestational stage of pregnancy. It will be understood by one skilled in the art that a period of gestation corresponds to a time-period elapsed since fertilization of a developing embryo or fetus. Thus, the stage of differentiation of a developing organ corresponds to the developmental stage of the embryo or fetus from which it is derived. Porcine and human gestational development have been extensively studied and characterized, and, as such, the ordinarily skilled artisan will possess the necessary expertise for suitably obtaining a porcine or human organ at a specific gestational stage so as to enable the practicing of the present invention.

Thus, according to an exemplary embodiment, the organ is obtained from a fetus at a gestational stage which enables optimal organ functionality and immuno-compatibility without teratoma formation. WO 2003/022123, WO 2004/078022, WO 2006/038211, WO 2006/077592 provide sufficient guidance for selecting the appropriate gestational stage for complying with these pre-requisites, each of which is hereby incorporated by reference in its entirety. For example, as described in the Examples section which follows, when using a porcine hepatic graft for practicing hepatic transplantation method of the present invention, the graft is derived from a porcine liver which may be at a developmental stage selected from a range of 25 to 56 days of gestation, at a developmental stage selected from a range of 26 to 56, at a developmental stage selected from a range of 27 to 56 days of gestation, at a developmental stage selected from a range of 28 to 56 days of gestation, at a developmental stage selected from a range of 28 to 42 days of gestation, at a developmental stage selected from a range of 27 to 29 days of gestation, or at a developmental stage of 28 days of gestation.

When using a porcine pancreatic tissue of the present invention, the graft is derived from a porcine pancreas which may be at a developmental stage selected from a range of about 42 to about 80 days of gestation, at a gestational stage of about 42 to about 56 days of gestation, or at a developmental stage of 42 days of gestation.

Likewise, when using a porcine splenic tissue of the present invention, the graft is derived from a porcine spleen which is at a developmental stage selected from a range of about 42 to about 80 days of gestation, at a gestational stage of about 42 to about 56 days of gestation, or at a developmental stage of 42 days of gestation.

According to an exemplary embodiment of the present invention, the transplanted organ is obtained from a human being, including a human fetus. For example, when using a human hepatic tissue, the organ is preferably derived from a human liver which is at a developmental stage selected from a range of 6 to 14 weeks of gestation, 6 to 13 weeks of gestation, 6 to 12 weeks of gestation, 6 to 11 weeks of gestation, 6 to 10 weeks of gestation, 6 to 9 weeks of gestation, 6 to 8 weeks of gestation, or 7 weeks of gestation.

The following table provides examples of the gestational stages of human and porcine grafts at which these can provide grafts which are essentially at corresponding developmental stages:

TABLE 1
Corresponding gestational stages of pigs and humans:

| Gestational stage | Gestational stage |
|---|---|
| of porcine graft (days) | of human graft (days) |
| 18 | 44 |
| 20 | 49 |
| 22 | 54 |
| 23 | 56-57 |
| 25 | 61-62 |
| 26 | 63 |
| 28 | 68-69 |
| 31 | 75 |
| 38 | 92 |
| 42 | 102 |
| 46 | 112 |
| 49 | 119 |
| 56 | 136 |
| 62 | 151 |
| 72 | 175 |
| 80 | 195 |
| 88 | 214 |

The gestational stage (in days) of a graft belonging to a given species which is at a developmental stage essentially corresponding to that of a porcine graft can be calculated according to the following formula: [gestational stage of porcine graft in days]/[gestational period of pig in days] × [gestational stage of graft of given species in days]. Similarly, the gestational stage (in days) of a graft belonging to a given species which is at a developmental stage essentially corresponding to that of a human graft can be calculated according to the following formula: [gestational stage of human graft in days]/[gestational period of humans in days] × [gestational stage of graft of given species in days]. The gestational stage of pigs is about 115 days and that of humans is about 280 days.

The present invention envisages that organs for transplantation are derived from species other than human or pig which are at stages of differentiation corresponding to the presently disclosed optimal gestational stages. Animals such as the major domesticated or livestock animals, and primates, which have been extensively characterized with respect to correlation of stage of differentiation with gestational stage may be suitable for practicing the present methods. Such animals include various mammalian species, such as, but are not limited to, bovines (e.g., cow), equids (e.g., horse), porcines (e.g. pig), ovids (e.g., goat, sheep), felines (e.g., Felis domestica), canines (e.g., Canis domestica), rodents (e.g., mouse, rat, rabbit, guinea pig, gerbil, hamster), and primates (e.g., chimpanzee, rhesus monkey, macaque monkey, marmoset) or human beings.

It will be appreciated that the organ according to the present invention may also be obtained from a postnatal organism. Thus, the organ may be obtained from an organism during the period beginning immediately after birth and extending for about six weeks.

Furthermore, according to the present teachings, the organ may be obtained from an adult, either a living or cadaver donor. If the organ is obtained from a cadaver donor, it is best to obtain the organ within 36-50 hours of death as to enable optimal chances of engraftment and functionality. In order to minimize rejection of transplanted organs, it will be appreciated that factors such as blood type and tissue type should be considered prior to transplantation.

Various common art methods may be employed to obtain an organ for transplantation.

Transplanting an organ of the present invention may be effected by transplanting the organ into any one of various anatomical locations, depending on the application. The organ may be transplanted into a homotopic anatomical location (a normal anatomical location for the organ transplant), or into an ectopic anatomical location (an abnormal anatomical location for the transplant). Depending on the application, the graft may be advantageously implanted under the renal capsule, or into the kidney, the testicular fat, the sub cutis, the omentum, the portal vein, the liver, the spleen, the heart cavity, the heart, the chest cavity, the lung, the pancreas and/or the intra abdominal space.

For example, a liver of the present invention may be transplanted into the liver, the portal vein, the renal capsule, the sub-cutis, the omentum, the spleen, and the intra-abdominal space. Transplantation of a liver into various anatomical locations such as these is commonly practiced in the art to treat diseases amenable to treatment via hepatic transplantation. Similarly, transplanting the pancreas of the present invention may be advantageously effected by transplanting the tissue into the portal vein, the liver, the pancreas, the testicular fat, the sub-cutis, the omentum, an intestinal loop (the subserosa of a U loop of the small intestine) and/or the intra-abdominal space.

Following transplantation of the organ into a subject according to the present teachings, it is advisable, according to standard medical practice, to monitor the growth functionality and immuno-compatability of the organ according to any one of various standard art techniques. For example, as described in the Example section below, the functionality of a pancreas transplant may be monitored following transplantation by standard pancreas function tests (e.g. analysis of serum levels of insulin) Likewise, liver transplant of the present invention may be monitored following transplantation by standard liver function tests (e.g. analysis of serum levels of albumin, total protein, ALT, AST, and bilirubin, and analysis of blood-clotting time). Structural development of the organ may be monitored via computerized tomography, or ultrasound imaging.

Depending on the transplantation context, in order to facilitate engraftment of the organ, the method may further advantageously comprise conditioning the subject with an immunosuppressive regimen prior to, concomitantly with, or following transplantation of the organ.

Examples of suitable types of immunosuppressive regimens include administration of immunosuppressive drugs, tolerance inducing cell populations, and/or immunosuppressive irradiation.

Ample guidance for selecting and administering suitable immunosuppressive regimens for transplantation is provided in the literature of the art (for example, refer to: Kirkpatrick C H. and Rowlands D T Jr., 1992. JAMA. 268, 2952; Higgins R M. et al., 1996. Lancet 348, 1208; Suthanthiran M. and Strom T B., 1996. New Engl. J. Med. 331, 365; Midthun D E. et al., 1997. Mayo Clin Proc. 72, 175; Morrison V A. et al., 1994. Am J. Med. 97, 14; Hanto D W., 1995. Annu Rev Med. 46, 381; Senderowicz A M. et al., 1997. Ann Intern Med. 126, 882; Vincenti F. et al., 1998. New Engl. J. Med. 338, 161; Dantal J. et al. 1998. Lancet 351, 623).

Preferably, the immunosuppressive regimen consists of administering at least one immunosuppressant agent to the subject.

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol. These agents may be administered individually or in combination.

It will be appreciated that the inventors of the present invention have uncovered a direct correlation between Factor VIII expression and transplant organ size. As clearly depicted in the Examples section hereinbelow, transplantation of porcine tissue (including spleen, pancreas and liver) into Factor VIII knock out mice leads to enlarged organ size compared to wild-type mice without effecting organ functionality.

It will be appreciated that any factors upstream, downstream or in physical association with Factor VIII (e.g. von Willebrand factor) may be modulated using the present teachings.

Thus, according to an embodiment of the present invention modulating transplant organ size comprises enhancing the transplant organ size. This may be effected using an agent capable of down-regulating an activity or expression of a coagulation factor or an effector thereof.

The phrase “coagulation factor” refers to a component of the coagulation cascade including, but not limited to, Factor VIII, Factor VIIIa, Factor V, Factor Va, Factor X, Factor Xa, Prothrombin, Thrombin, Fibrinogen, Factor XIII and Factor XIIIa.

The phrase “an effector of a coagulation factor” refers to a downstream biological pathway regulated by a product of the coagulation cascade such as Protease-Activated Receptor (PAR).

The term “Factor VIII” as used herein refers to coagulation Factor VIII or mimetics thereof such as set forth in GenBank Accession Nos. NP_{—}000123 (SEQ ID NO: 6), NM_{—}000132 (SEQ ID NO: 7) and NP_{—}063916 (SEQ ID NO: 8).

The term “Factor Xa” as used herein refers to coagulation Factor X or mimetics thereof such as set forth in GenBank Accession Nos. NM_{—}000504 (SEQ ID NO: 9) and NP_{—}000495 (SEQ ID NO: 10).

The term “Thrombin” as used herein refers to coagulation Factor IIa or mimetics thereof such as set forth in GenBank Accession Nos. NM_{—}000506 (SEQ ID NO: 11) and NP_{—}000497 (SEQ ID NO: 12).

The phrase “Protease-Activated Receptor (PAR)” as used herein refers to the seven transmembrane G-protein-coupled receptor, which is expressed throughout the body (e.g. on platelets, endothelial cells, myocytes and neurons) and is typically activated by the action of serine proteases such as thrombin. Examples of PAR receptors include, but are not limited to, PAR1 e.g. as set forth in GenBank Accession Nos. NM_{—}001992 and NP_{—}001983, PAR2 e.g. as set forth in GenBank Accession Nos. NM_{—}005242 and NP_{—}005233, PAR3 e.g. as set forth in GenBank Accession Nos. NM_{—}004101 and NP_{—}004092 and PAR4 e.g. as set forth in GenBank Accession Nos. NM_{—}003950 and NP_{—}003941.

It will be appreciated that the coagulation factor Factor V includes e.g. GenBank Accession Nos. NM_{—}000130 and NP_{—}000121, the coagulation factor Fibrinogen includes e.g. GenBank Accession Nos. NM_{—}000509, NM_{—}000508, NM_{—}005141, NP_{—}000500, NP_{—}000499 and NP_{—}005132, and the coagulation factor Factor XIII includes e.g. GenBank Accession Nos. NM_{—}001994, NM_{—}000129, NP_{—}001985 and NP_{—}000120.

It will be appreciated that activators of Factor VIII can be modulated according to the present teachings. Examples include, but are not limited to, Factor XII (e.g. GenBank Accession Nos. NM_{—}000505 and NP_{—}000496), Factor XIIa, Factor XI (e.g. GenBank Accession Nos. NM_{—}000128 and NP_{—}000119), Factor XIa, Factor IX (e.g. GenBank Accession Nos. NM_{—}000133 and NP_{—}000124), Factor IXa, protein C (e.g. GenBank Accession Nos. NM_{—}000312 and NP_{—}000303), Von Willebrand factor (vWF, e.g. GenBank Accession Nos. NM_{—}000552 and NP_{—}000543), Factor VII (e.g. GenBank Accession Nos. NM_{—}000131, NM_{—}019616, NP_{—}000122 and NP_{—}062562) and Factor VIIa.

The phrase “activity or expression of a coagulation factor or an effector thereof” as used herein refers to the activity of the coagulation factor or an effector thereof on modulation of transplant organ size and may be independent of the coagulation activity of the factor.

Thus, enhancement in transplant organ size is achieved by down-regulating the expression level and/or activity of a coagulation factor or an effector thereof in the subject. Down-regulating the expression level and/or activity of a coagulation factor or an effector thereof is preferably effected so as to maximally decrease the expression level and/or activity of the coagulation factor or an effector thereof in the subject, so as to achieve optimal enhancement in transplant organ size. Down-regulating the expression level and/or activity of a coagulation factor or an effector thereof can be achieved in any of various ways.

Downregulation of a coagulation factor or an effector thereof can be effected on the genomic and/or the transcript level using a variety of molecules which interfere with transcription and/or translation (e.g., RNA silencing agents, Ribozyme, DNAzyme and antisense), or on the protein level using e.g., antagonists, enzymes that cleave the polypeptide and the like.

Following is a list of agents capable of down-regulating expression level and/or activity of a coagulation factor or an effector thereof.

Downregulation of a coagulation factor or an effector thereof can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to down-regulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl. Acad. Sci. USA. 2002; 99:1443-1448; Tran N., et al., FEBS Lett. 2004; 573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392.doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target). The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to a miRNA, rather than triggering RNA degradation.

Synthesis of RNA silencing agents suitable for use with the present invention can be effected as follows. First, the coagulation factor (e.g. Factor VIII) mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, a suitable Factor VIII siRNA can be the siRNA ID s4940, s4941 or s4942 (Ambion Inc., Austin, Tex.).

A suitable Factor X siRNA can be e.g. human F10 Chimera RNAi (Abnova Corporation) and human F10 shRNA (OriGene Technologies).

A suitable Thrombin siRNA can be e.g. human Thrombin R siRNA (Santa Cruz Biotechnology, Inc.).

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide”. As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

Another agent capable of downregulating a coagulation factor (e.g. Factor VIII) is a DNAzyme molecule capable of specifically cleaving an mRNA transcript or DNA sequence of the coagulation factor (e.g. Factor VIII). DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a coagulation factor or an effector thereof can also be effected by using an antisense polynucleotide capable of specifically hybridizing with an mRNA transcript encoding a coagulation factor or an effector thereof (e.g. Factor VIII, Factor X and Thrombin).

Design of antisense molecules which can be used to efficiently downregulate a coagulation factor or an effector thereof must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published [Matveeva et al., Nature Biotechnology 16: 1374-1375 (1998)].

For example, a suitable antisense oligonucleotides targeted against the Factor VIII mRNA (which is coding for the Factor VIII protein) would be of the following sequences:

(SEQ ID NO: 1)
5′ G T C C A C T T G C A G C C A C T C T
T 3′,
(SEQ ID NO: 2)
5′ G T C C A C T T G C A G C C A C T C T
3′,
(SEQ ID NO: 3)
5′ G T C C A C T T G C A G C C A C T C T
T T 3′,
(SEQ ID NO: 4)
5′ G C T T T A C T C T C C A T T C C C A
3′,
or
(SEQ ID NO: 5)
5′ T G C T T T A C T C T C C A T T C C C
A 3′.

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for down-regulating expression of known sequences without having to resort to undue trial and error experimentation.

Another agent capable of down-regulating a coagulation factor or an effector thereof is a ribozyme molecule capable of specifically cleaving an mRNA transcript encoding a coagulation factor (e.g. Factor VIII). Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of Factor VIII gene and/or genes of other coagulation factors in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | |
|---|---|
| oligo | 3′--A G G T |
| duplex | 5′--A G C T |
| duplex | 3′--T C G A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the Factor VIII regulatory region (or the regulatory region of other coagulation factors or effectors thereof) a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Downregulation of a coagulation factor or an effector thereof can also be effected at the protein level using e.g., antagonists, enzymes. For example, Factor VIII can be down-regulated by, for example, Factor VIII antagonists [e.g. TB-402 (Thromb-X NV)] or Factor VIII inhibitory peptide (e.g. Factor VIII neutralizing antibody). Downregulation of Factor X can be effected using, for example, Clexane, JTV-803 or Fondaparinux. Downregulation of Thrombin can be effected using, for example, Clexane, Dabigatran, Hirudin, Bivalirudin, Lepirudin, Desirudin, Argatroban, Melagatran or ximelagatran.

Another agent which can be used along with the present invention to down-regulate a coagulation factor or an effector thereof is a molecule which prevents a coagulation factor's (e.g. Factor VIII) activation or substrate binding. Such a molecule may comprise an antibody which specifically binds Factor VIII, as for example, sc-73597 [Santa Cruz Biotechnology] or F4.55, F4.77, F4.264, F4.115 and F4.415 [Sola et al., PNAS (1982) 79 (1) 183-187] Likewise, antibodies which specifically target Factor X (e.g. ab61361, Abcam) or Thrombin (e.g. sc-59716, sc-80590, sc-73475, sc-59717, sc-59718, sc-65961, Santa Cruz Biotechnology) may be used according to the present teachings.

According to a specific embodiment of the present invention, synthetic peptides or antibodies which inhibit PARs (directed at e.g. PAR1, PAR2, PAR3 or PAR4) may also be used to downregulate PAR signaling, such as for example, the PAR1 agonist TFLLR-NH2 (SEQ ID NO: 13), the PAR4 agonist AYPGKF-NH2 (SEQ ID NO: 14), the palmitoylated peptides pal-RCLSSSAVANRS (SEQ ID NO: 15, PAR1 antagonist) and pal-SGRRYGHALR (SEQ ID NO: 16, PAR4 antagonist). Such peptide antagonists may be generated by any method known to one of ordinary skill in the art, such as by solid-phase peptide synthesis using in situ neutralization/HBTU by Hadar Biotec, Israel.

It will be appreciated that downregulation of a coagulation factor or an effector thereof can also be effected by up-regulating the activity or expression of antithrombin or Protein C.

It will be appreciated that according to the present teachings Vitamin K levels may also be modulated to enhance or decrease organ size.

It will be appreciated that in order to increase organ size (e.g. spleen), G-CSF may be administered prior to, concomitantly with, or following administration of the above described agents (e.g. Clexane) Likewise, other growth factor and/or cytokines may be administered to the subject to modulate organ size including, but not limited to, Hepatocyte growth factor (HGF) and Keratinocyte growth factor (KGF).

In some instances decreasing organ size may be desirable while maintaining functionality. For instance, kidney transplantation from an adult to an infant may be desired or decreasing splenomegaly of a transplanted organ. It will be appreciated that according to the present teachings, decreasing transplant organ size is achieved by up-regulating the expression level and/or activity of a coagulation factor or an effector thereof in the subject. Up-regulating the expression level and/or activity of a coagulation factor or an effector thereof is preferably effected so as to maximally increase the expression level and/or activity of a coagulation factor or an effector thereof in the subject, so as to achieve optimal decrease in transplant organ size. Up-regulating the expression level and/or activity of a coagulation factor or an effector thereof can be achieved in any of various ways.

For example, upregulation Factor VIII can be effected by administering to the subject human Factor VIII (e.g. plasma-derived Factor VIII), recombinant Factor VIII (e.g. rFVIII, Bayer Biological Products, EU), porcine factor VIII (e.g. HYATE:C), activated prothrombin complex (e.g. APCC, Baxter Healthcare, US) and desmopressin (e.g. DDAVP, Stimate, Minirin). Upregulation of Factor X or Xa may be achieved by administering to the subject the factors per se, available from CalBiochem, La Jolla, Calif. Upregulation of Thrombin may be achieved by administering to the subject Prothrombin or Thrombin, available from CalBiochem, La Jolla, Calif.

It will be appreciated that decreasing an organ size can also be effected by down-regulating the activity or expression of anti-thrombin.

According to an exemplary embodiment of the present invention, modulating the expression level and/or activity of a coagulation factor or an effector thereof may be effected prior to, concomitantly with or following transplantation of an organ. Thus, modulating the expression level and/or activity of a coagulation factor or an effector thereof is effected so as to maximally enable organ engraftment into the subject with minimal organ failure.

Each of the agents used for up-regulating or down-regulating coagulation factor or an effector thereof described hereinabove can be administered to the subject per se or as part of a pharmaceutical composition which also includes a physiologically acceptable carrier. The purpose of a pharmaceutical composition is to facilitate administration of the active ingredient to an organism.

It will be appreciated that the pharmaceutical composition may further comprise an immunosuppressive agent as described in detail hereinabove.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the coagulation factor or an effector thereof thereof upregulating or downregulating agents accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of an aggregate of cells having a similar structure and/or a common function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (coagulation factor or an effector thereof upregulating or downregulating agents) effective to modulate transplant organ size of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide adequate levels of the active ingredient as to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The modulating factors will be given for a sufficient amount of time to enable modulation of organ transplant size without compromising blood coagulation levels (e.g. bleeding or blood clot formation) in the subject. Thus, it is advisable to draw a base-line blood sample from each subject prior to administration of the modulating agents of the present invention. Furthermore, once a subject received modulating factors, it is advisable that they return for follow-up evaluation, which include, for example, hematologic and chemical tests for safety.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As mentioned hereinabove, the teachings of the present invention can be employed to treat essentially any disorder which is amenable to treatment via organ transplantation. Thus, the teachings of the present invention can be utilized for treating any disorder including hepatic disorders [including, without limitation, hepatitis C infection, hepatobiliary malignancies such as hepatocellular carcinoma, cirrhosis, primary sclerosing cholangitis, alcoholic liver disease, hepatitis B, drug/toxin-induced hepatotoxicity, hepatic vascular injury, autoimmune hepatitis, blunt hepatic trauma, liver damage associated with inborn errors of metabolism, urea cycle defects, hypercholesterolemia, glycogen storage disease, primary hyperoxaluria type I, cryptogenic cirrhosis, Crigler-Najjar syndrome type I, congenital hepatic fibrosis, Neimann-Pick disease, primary biliary cirrhosis, amyloidosis, biliary atresia, hepatoblastoma, Alagille syndrome, hemangioendothelioma, cholestasis, acute/fulminant liver failure, Budd-Chiari syndrome, alpha-1-antitrypsin deficiency, Wilson disease, hemochromatosis, tyrosinemia, disorders of porphyrin metabolism such as protoporphyria, cystic fibrosis, malignant neoplasm of intrahepatic bile ducts, lipidoses, disorders of copper metabolism, disorders of purine and pyrimidine metabolism, disorders of bilirubin excretion, mucopolysaccharidosis, congenital factor VIII disorder, congenital factor IX disorder, necrosis of liver, alcoholic fatty liver, sequelae of chronic liver disease, disorders of gallbladder, bile duct obstruction, biliary atresia, perinatal jaundice due to hepatocellular damage, portal vein thrombosis (PVT), hemophilia and lysosomal storage diseases/enzyme deficiencies such as Gaucher disease]; renal disorders [including, without limitation, acute kidney failure, acute nephritic syndrome, analgesic nephropathy, atheroembolic kidney disease, chronic kidney failure, chronic nephritis, congenital nephrotic syndrome, end-stage kidney disease, Goodpasture's syndrome, IgM mesangial proliferative glomerulonephritis, interstitial nephritis, kidney cancer, kidney damage, kidney infection, kidney injury, kidney stones, lupus nephritis, membranoproliferative glomerulonephritis I, membranoproliferative glomerulonephritis II, membranous nephropathy, necrotizing glomerulonephritis, nephroblastoma, nephrocalcinosis, nephrogenic diabetes insipidus, IgA-mediated nephropathy, nephrosis, nephrotic syndrome, polycystic kidney disease, post-streptococcal glomerulonephritis, reflux nephropathy, renal artery embolism, renal artery stenosis, renal papillary necrosis, renal tubular acidosis type I, renal tubular acidosis type II, renal underperfusion and renal vein thrombosis]; pancreatic disorders [including, without limitation, Type 1 Diabetes, Type 2 Diabetes, Pancreatitis, Pancreatic Cancer and Pseudocysts of the Pancreas]; splenic disorders [including, without limitation, splenomegaly, Gaucher disease and Sarcoidosis]; heart disorders [including, without limitation, heart failure, coronary artery disease, viral heart infections, damaged heart valves and damaged heart muscle]; lung disorders [including, without limitation, Cystic Fibrosis, Pulmonary arterial hypertension (PAH), Chronic Obstructive Pulmonary Disease (COPD), Pulmonary Fibrosis or Interstitial Lung Disease (ILD), Bronchiectasis, Pulmonary Hypertension, Sarcoidosis and Lymphangioleiomyomatosis] and intestinal disorders [including, without limitation, Short Gut Syndromes, Malabsorption Syndromes, Motility Disorders and Tumors of the Intestinal Mesentery].

It is expected that during the life of a patent maturing from this application many relevant upregulating or downregulating agents for coagulation factors or effectors thereof will be developed and the scope of the term coagulation factor or an effector thereof upregulating or downregulating agents is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Involvement of Factor VIII in the Control of Transplant Organ Size During Transplantation of Embryonic Porcine Grafts into SCID Mice

Materials and Experimental Procedures

Animals

All animals were maintained under conditions approved by the Institutional Animal Care and Use Committee at the Weizmann Institute. The study protocol was approved by the ethics committees at Kibbutz Lahav and the Weizmann Institute.

8-10 week old immune deficient NOD-SCID or factor VIII KO-SCID mice (Weizmann Institute Animal Breeding Center, Rehovot, Israel) were used as hosts for the transplantation studies. To obtain immunodeficient hemophilic mice (designated factor VIII KO-SCID), FVIII-deficient mice were crossed with SCID mice, as was previously described [Aronovich, A. et al., Proc Natl Acad Sci USA (2006) 103: 19075-80]. All mice were kept in small cages (up to five animals per cage) and fed sterile food.

In addition, immune deficient RAG^{-/-} mice or RAG^{-/-} FVIII KO mice were used as hosts for the transplantation studies. To obtain RAG^{-/-} hemophilic mice (designated RAG^{-/-} FVIII KO mice), FVIII mutation was introduced into RAG^{-/-} mice, as was previously described. All mice were kept in small cages (up to five animals per cage) and fed sterile food.

Pig embryos were obtained from the Lahav Institute of Animal Research (Kibbutz Lahav, Israel). Pregnant sows were operated on at embryonic days 28 (E28), for liver tissue, and 42 (E42), for spleen and pancreatic tissues, under general anesthesia. Warm ischemia time was less than 10 minutes and the embryos were transferred to cold PBS. Spleen, pancreas and liver precursors for transplantation were extracted under a light microscope and were kept in sterile conditions at 4° C. in RPMI 1640 (Biological Industries, Beit HaEmek, Israel) prior to transplantation. Cold ischemia time until transplantation was less than 2 hours.

Mouse embryos were obtained from C57BL/6 pregnant female mice. Pregnant mice were operated on at embryonic days 15 (E15), for spleen tissue, and 16 (E16), for liver and pancreatic tissues, under general anesthesia. Warm ischemia time was less than 10 minutes and the embryos were transferred to cold PBS. Spleen, pancreas and liver precursors for transplantation were extracted under a light microscope and were kept in sterile conditions at 4° C. in RPMI 1640 (Biological Industries, Beit HaEmek, Israel) prior to transplantation. Cold ischemia time until transplantation was less than 2 hours.

Transplantation Procedure

Transplantation of pig precursors was completed as previously described [Dekel, B. et al., Nat Med (2003) 9, 53-60]. Briefly, transplantation of the embryonic precursors was performed under general anesthesia (2.5% 2,2,2-Tribromoethanol, 97% in PBS, 10 ml/kg intraperitoneally). Host kidney was exposed through a left lateral incision. A 1.5-mm incision was made at the caudal end of the kidney capsule and donor precursors were grafted under the kidney capsule in fragments 1-2 mm in diameter.

Morphometric Analysis

Three months post transplantation E42 pancreatic grafts were formalin fixed and embedded in paraffin. Consecutive 40 μm sections were cut and stained. The areas of interest were quantified using the Image Pro program (Media Cybernetics).

ELISA Measurements of Pig Insulin and Albumin

A porcine/human insulin kit (Catalog No. K6219, DAKO), in which the primary pig anti-insulin antibody does not cross-react with mouse insulin, was used to follow pig insulin levels according to the manufacturer's instructions.

Pig albumin in mouse serum was measured by a standard ELISA procedure using primary goat anti-pig albumin antibody (human, mouse, and bovine absorbed), affinity purified, and secondary pig specific horseradish-peroxidase conjugated antibody (Catalog Nos. A100-210A and A100-210P, Bethyl).

Histochemistry

Histochemistry included hematoxylin/eosin (H&E) and periodic acid/Schiff (PAS). For immunohistochemical labeling, the following antibodies were used: goat anti-pig albumin antibody (Bethyl Laboratories, Montgomery, Tex.), rabbit anti-human glucagon (DAKO), guinea pig anti-rabbit insulin (DAKO) and mouse anti porcine CD31 (Serotec, Enco Scientific Services Ltd Israel).

Paraffin sections (4μ) were xylene deparaffinized and rehydrated. Endogenous peroxidase was blocked with 0.3% H2O2 in 70% methanol for 10 minutes. Antigen-retrieval procedures were performed according to the glucagon antibody manufacturer's instructions. After blocking, both paraffin sections and 6-μ cryosections were incubated with specific first antibody for 60 minutes. Detection of antibody binding was performed by using the following secondary reagents: DAKO peroxidase EnVision system for the detection of mouse and rabbit antibodies and Sigma biotinylated anti-goat antibody (followed by extra avidin peroxidase reagent) for goat antibodies. In all cases, diaminobenzidine was used as a chromogen.

Immunofluorescence protocols were applied using secondary antibodies: donkey anti mouse Texas red (Jackson), donkey anti rat conjugated CY2 or Texas red (Jackson).

Statistical Analysis

Comparisons between groups were evaluated by the Student's t-test. Data were expressed as mean±SD and were considered statistically significant at p values of 0.05 or less.

Results

Porcine Spleen

The inventors of the present invention have unexpectedly discovered that transplanted pig embryonic tissues grew to a larger size in immunodeficient hemophilic (Factor VIII KO-SCID) recipient mice in comparison non-hemophilic NOD-SCID mice. As illustrated in FIGS. 1A-B, three months post transplant of pig embryonic spleen tissue in a Factor VIII KO SCID recipient mouse, the implanted spleen displayed a typical oversize versus the size of the implant grown in a factor VIII positive SCID recipient. The total average weight of the implants was 6.78±2.16 gr for Factor VIII KO-SCID mice compared to 1.46±0.82 gr for SCID mice (FIG. 1C), suggesting enhancement of spleen size by a factor of 4 (p<0.05). Histological examination of the growing spleen implants revealed normal growth, development and vascularization patterns comparable to those found in the corresponding factor VIII wild type mice (FIGS. 1D-G), ruling out the potential induction of a malignant process. Together this data indicated a potential involvement of mouse factor VIII in size control of pig embryonic spleen implants.

Porcine Liver and Pancreas

To generalize this finding, inventors further evaluated the growth of pig embryonic liver and pancreas precursor tissues harvested at a previously defined optimal ‘window’, namely E28 and E42, respectively [Eventov-Friedman, S. et al., Proc Natl Acad Sci USA (2005) 102, 2928-33]. The growth of pancreatic tissues was determined in the SCID recipient mice by monitoring blood levels of pig insulin using a specific ELISA previously shown to correlate with implant size [Eventov-Friedman, S. et al., supra]. As can be seen in FIG. 2A, pig insulin blood levels were markedly enhanced in Factor VIII KO SCID mice compared to Factor VIII positive SCID mice, reflecting a growth advantage in the Factor VIII KO recipients. However, these differences in insulin levels could be attributed to differences in functionality of β-cells rather than to the total number of β-cells. Therefore, the total volume of the implants as well as the fraction of β-insulin positive cells (out of the total volume) were examined using morphometric analysis (FIG. 2B). As is illustrated in FIG. 2B, enhancement of pig pancreas size was found to closely correspond to the difference in insulin blood levels. Thus, the total volume of insulin positive cells was 0.75±0.003 mm³ in factor VIII SCID versus 0.23±0.003 mm³ in non-hemophilic SCID recipients, respectively, suggesting an overall enhancement of implant size at least by a factor of three (p<0.05).

Similarly to previously described results [Eventov-Friedman, S. et al., PLoS Med (2006) 3, e215], E42 pancreatic tissue implanted into NOD-SCID mice were shown to predominantly comprise endocrine tissue with minimal exocrine activity. Only a minimal number of exocrine cells were detected in the E42 graft three months after transplantation, while most of the cells were of the endocrine lineage. Moreover, the endocrine compartment architecture of the growing pig pancreas was similar for both wild type (FIG. 2C) and Factor VIII KO recipient mice (FIG. 2D).

A significant enhancement in implant size (by at least a factor of two) was also established upon implantation of embryonic pig liver into factor VIII KO SCID mice, as evaluated by ELISA for pig albumin blood levels (FIG. 3A). Again, despite the enhanced growth, the growing pig liver exhibited similar architecture in both types of recipients (FIGS. 3B-G).

Taken together, these results strongly suggest that mouse factor VIII plays a critical role in controlling the size of embryonic pig implants.

Implantation of Embryonic Mouse Spleen, Pancreas and Liver into Factor VIII KO SCID Mice Exhibit No Enhancement of Size

It is nevertheless possible that the enhancement of organ size following implantation of pig embryonic spleen, pancreas and liver, might be related to early events associated with graft accommodation and vasculature formation, which might potentially differ in Factor VIII KO versus non-hemophilic SCID recipients and could be independent of the origin of the donor tissue. To address this possibility, similar implantation experiments were repeated using embryonic tissues from C57BL/6 mouse donors. In contrast to the pig implants, no differences in organ size were found between SCID factor VIII KO and non-hemophilic SCID recipients following implantation of mouse E15-16 gestational age tissues (data not shown).

Spleen grafts of mouse origin exhibited an average size of 1.5±0.35 mm³ three months post transplantation in both SCID and SCID Factor VIII KO recipients. Similar results were obtained for mouse embryonic pancreas and liver transplants (data not shown). Furthermore, no differences in size were found following transplantation of embryonic mouse tissues obtained from hemophilic donors into factor VIII KO-SCID versus non-hemophilic SCID mice. Thus, these results suggest that while the final size of heterologous embryonic pig implants is affected by the presence or absence of mouse Factor VIII, embryonic mouse implants attain their final size regardless of the presence of mouse factor VIII.

RAG−/− Factor VIII KO Mice also Exhibit Enhanced Implant Size Following Transplantation of Pig Embryonic Tissues

Prkdc^{scid} (commonly referred to as SCID) is a spontaneously occurring mutation in chromosome 16. Furthermore, the Prkdc^{scid} mutation was backcrossed onto the NOD/ShiLt background to obtain the NOD-SCID mice. NOD-SCID mice are characterized by an absence of functional T cells and B cells, lymphopenia, hypogammaglobulinemia and a normal hematopoietic microenvironment. On the other hand, hemophilic (Factor VIII KO) mice are homozygous for the targeted, X chromosome-linked mutant allele, by a neo cassette which was used to disrupt exon 16 of Factor VIII gene. As previously described [Aronovich A. et al., Proc Natl Acad Sci USA (2006) 103: 19075-80], by backcrossing these two strains a new strain was developed of Factor VIII KO mice on a background of NOD-SCID mice.

Despite the fact that the SCID mutation in NOD mice is on chromosome 16 and hemophilia is a chromosome X linked mutation, a risk of chromosome translocation exists, as in the case of Philadelphia chromosome abnormality that is associated with chronic myelogenous leukemia. Therefore, in order to rule out potential artifacts due to genetic abnormalities in NOD-SCID FVIII KO mice, inventors have attempted to introduce the Factor VIII KO mutation into a different SCID mouse, namely, SCID with a RAG^{-/-} background which was previously shown to exhibit a targeted mutation on chromosome 2, associated with a “non-leaky” severe combined immune deficiency. Thus, a new RAG^{-/-} FVIII KO colony was established.

As can be seen in FIGS. 3H-I, RAG^{-/-} FVIII KO recipients of an E42 pig spleen implant exhibit at twelve weeks post transplantation an oversized spleen implant compared to their non-hemophilc RAG^{-/-} counterparts.

### Example 2

### Involvement of Factor VIII in Splenomegaly During G-CSF Treatment

Materials and Experimental Procedures

Animals

8-10 week old immune deficient NOD-SCID or factor VIII KO-SCID mice (Weizmann Institute Animal Breeding Center, Rehovot, Israel) were used for G-CSF studies. To obtain immunodeficient hemophilic mice (designated factor VIII KO-SCID), FVIII-deficient mice were crossed with SCID mice, as was previously described [Aronovich, A. et al., Proc Natl Acad Sci USA (2006) 103: 19075-80].

8 to 10 week old immune competent C57BL mice and C57BL hemophilic (C57BL Hem F8) mice were used for the G-CSF studies.

All mice were kept in small cages (up to five animals per cage) and fed sterile food.

G-CSF Treatment

8-10 weeks old NOD-SCID or Factor VIII KO SCID mice were treated by daily subcutaneous injections of recombinant human G-CSF (Neupogen, Amgen) at a dose of 250 μg per kg per day for 7 days. To determine the spleen weight, 7 days after the initiation of G-CSF treatment, mice were euthanized and spleens were harvested.

Factor VIII Infusion

Hu Factor VIII was infused into Factor VIII KO SCID mice by osmotic pumps (Azlet pump model 1003D, 1 μl/hr rate, 100 μl total capacity with a continuous delivery for 3 days). Thus, 60 IU Hu factor VIII was dissolved in 100 μl PBS and delivered by the 1003D pump for 3 days. The pump was administrated into the peritoneal cavity. After 3 days, a similar new pump was administrated. Initial concentration of the Hu Factor VIII was calibrated based on Hu Factor VIII half life and its clearance rate [Mordenti, J. et al., Toxicol Appl Pharmacol (1996) 136: 75-81].

Statistical Analysis

As described in detail in Example 1, hereinabove.

Results

Factor VIII KO SCID Mice Exhibit Enhanced G-CSF Induced Splenomegaly

As depicted in detail above, Factor VIII deficiency had no effect on organ size of mouse embryonic transplants, thus suggesting that the role of Factor VIII may be limited to a checkpoint of excessive growth which only operates upon implantation of tissues from a larger organism, such as a pig. More specifically, it is possible that Factor VIII is involved in interference in expression and/or activity of a putative survival factor and thereby acts to define the maximum tolerable tissue mass. Thus, assuming that mouse and pig embryonic implants are endowed with stem cell pools of different sizes prior to transplantation, they are likely to exhibit different organ size upon completion of growth and differentiation in the mouse recipients (FIGS. 4A-B). Consequently, in hemophilic mice (e.g. SCID Factor VIII KO mice) lacking the potential inhibitory activity (i.e. overgrowth checkpoint) mediated by Factor VIII, the size of the pig transplanted organ size is larger, while mouse implants growing to the expected mouse size do not exhibit excessive growth and therefore are not subject to Factor VIII control (FIG. 4C).

It could be argued that the role of Factor VIII might be limited to the transplantation setting and therefore might not have a true physiological role in the intact animal. To test the potential control by Factor VIII on oversized organs in a more physiological setting, inventors have further tested the role of Factor VIII in controlling G-CSF mediated splenomegaly, known to occur within 7 days of infusion of this agent [Takamatsu, Y. et al., Transfusion (2007) 47, 41-9; Platzbecker, U. et al., Transfusion (2001) 41, 184-9]. As is illustrated in FIGS. 5A-B, treatment of Factor VIII KO SCID mice with G-CSF was associated with a significant enhancement in spleen size (i.e. splenomegaly) compared to non-hemophilic mice. Thus, the average spleen weight in the former group was 2.6 fold larger (314.89±121.51 mg, compared to 118.5±25.56 mg in the latter group, P<0.0001).

The capacity of exogenous Factor VIII infusion to inhibit the enhanced splenomegaly in SCID FVIII KO mice under G-CSF stimulation, is shown in FIG. 5B. Considering that the half life of Hu Factor VIII in the blood of infused mice was only about four hours, a continuous administration of Factor VIII (by osmotic pumps) was used in these mice. Although only about 10% of physiologically circulating Factor VIII was detected in the plasma of the infused mice, these SCID FVIII KO recipients exhibited reduced splenomegaly under G-CSF stimulation, compared to mice not receiving Factor VIII (FIG. 5B, P=0.033).

Enhancement of G-CSF Splenomegaly in Fully Immune Competent Factor VIII KO C57BL/6 Mice

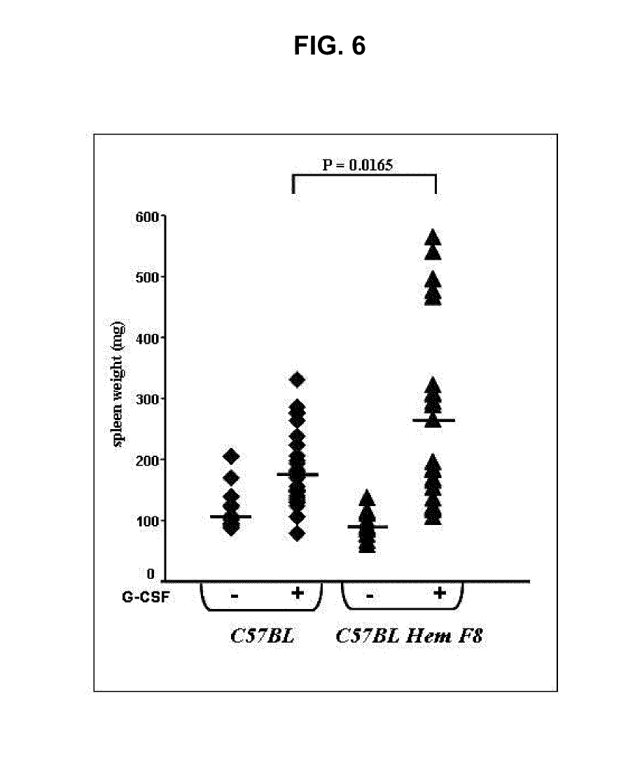

While investigation of implant size upon transplantation of pig embryonic tissue required the use of Factor VIII KO hemophilic SCID mice, the SCID background is not necessary for the evaluation of splenomegaly in G-CSF treated mice. Indeed, examination of the splenomegaly revealed a role for Factor VIII in oversized control also in fully immune competent Factor VIII KO C57BL/6 hemophilic mice (FIG. 6). Examination of the enlarged spleens revealed normal growth and development comparable to that found in the Factor VIII wild type counterpart mice, without any indication of malignancy.

As can be seen in FIG. 6, treatment with G-CSF of C57BL hemophilic (Hem) F8 mice was associated with significantly enhanced splenomegaly, compared to that found in the non-hemophilic counterparts (average spleen weight in the former group was 256.03±149.86 mg, compared to 181.22±61.43 mg in the latter group, P=0.0165), upon infusion of the same doses of G-CSF.

Taken together these results suggest that Factor VIII is involved in regulation of organ size in situations by which there is a drive for oversized growth, either due to large stem cell pool as in the case of embryonic organ transplantation (from a large transplant donor) or upon infusion of a cytokine such as G-CSF.

### Example 3

### Factor VIII Affects Organ Size Through Cell Growth, Proliferation and Apoptosis Genes

Materials and Experimental Procedures

Animals

As described in detail in Example 1, hereinabove.

Transplantation Procedure

As described in detail in Example 1, hereinabove.

DNA Microarray Analysis:

Array Processing

All experiments were performed using Affymetrix porcine genome oligonucleotide arrays, as described at: http://www(dot)affymetrix(dot)com/support/technical/datasheets/porcine_datasheet(dot)pdf.

Total RNA from each sample was used to prepare biotinylated target RNA, using minor modifications as recommended by the manufacturer at: http://www(dot)affymetrix(dot)com/support/technical/manual/expression_manual(dot)affx.

Briefly, 5 μg of mRNA was used to generate first-strand cDNA by using a T7-linked oligo(dT) primer. After second-strand synthesis, in vitro transcription was performed with biotinylated UTP and CTP (Affymetrix), resulting in approximately 300-fold amplification of RNA.

The target cDNA generated from each sample was processed as per manufacturer's recommendation using an Affymetrix GeneChip Instrument System: http://www(dot)affymetrix(dot)com/support/technical/manual/expression_manual(dot)affx.

Briefly, spike controls were added to 15 μg fragmented cRNA before overnight hybridization. Arrays were then washed and stained with streptavidin-phycoerythrin, before being scanned on an Affymetrix GeneChip scanner. A complete description of these procedures is available at: http://www(dot)affymetrix(dot)com/support/technical/manual/expression_manual(dot)affx.

Additionally, quality and amount of starting RNA was confirmed using an agarose gel. After scanning, array images were assessed by eye to confirm scanner alignment and the absence of significant bubbles or scratches on the chip surface. 3′/5′ ratios for GAPDH and beta-actin were confirmed to be within acceptable limits (3.16-3.4 and 0.38-0.4), and BioB spike controls were found to be present on all chips, with BioC, BioD and CreX also present in increasing intensity. When scaled to a target intensity of 150 (using Affymetrix MAS 5.0 array analysis software), scaling factors for all arrays were within acceptable limits (1.62-1.69), as were background, Q values and mean intensities. Details of quality control measures can be found at:

http://www(dot)ncbi(dot)nlm(dot)nih(dot)gov/geo/ or at

http://eng(dot)sheba(dot)co(dot)il/genomics;

http://www(dot) affymetrix(dot)com/support/technical/datasheets/porcine_datasheet(dot)pdf;

http://www(dot)affymetrix(dot)com/support/technical/manual/expression_manual(dot)affx;

http://www(dot)affymetrix(dot)com/support/technical/manual/expression_manual(dot)affx.

Data Analysis:

The probe sets contained in the Affymetrix porcine genome oligonucleotide array signals were calculated using Mas 5 algorithm. Pig implants affected by the difference in mouse Factor VIII expression (in the different SCID recipients) were compared. The comparison generated a list of “active genes” representing probe sets changed by at least 2 fold as calculated from the MAS 5 Log Ratio values(LR>=1 or LR<=−1) and detected as “Increased” or as “Decrease”(I or D, p-value 0.0025) or “Marginal Increased” or as or “Marginal Decrease” (MI or MD, p-value 0.003) in all treated sample as compared to all the control samples in at least one time point. This list excluded up-regulated genes in all treated samples with signals lower than 20 or detected as absent, and down regulated gene with base line signals lower than 20 and detected as absent in the control samples.

For further filtering we used the probe sets changed by at least 2 fold (between signals) between the x treated samples at x.

Hierarchical clustering was performed using Spotfire DecisionSite for Functional Genomics (Somerville, Mass.).

Genes were classified into functional groups using the GO annotation tool [Dennis G Jr, Sherman B T, Hosack D A, Yang J, Gao W, Lane H C, Lempicki R A. DAVID: Database for Annotation, Visualization, and Integrated Discovery. Genome Biology 2003) 4(5)) P3].

http://apps1(dot)niaid(dot)nih(dot)gov/David/publications/DAVID(dot)pdf

http://apps1(dot)niaid(dot)nih(dot)gov/David/upload(dot)asp.

Over-representation calculations were done using Ease [Douglas A Hosack, Glynn Dennis Jr, Brad T Sherman, H Clifford Lane, Richard A Lempicki. Identifying Biological Themes within Lists of Genes with EASE. Genome Biology (2003) 4(6):P4].

http://genomebiology(dot)com/2003/4/6/P4.

Functional classifications with an “Ease score” lower than 0.05 were marked as over represented.

Statistical Analysis

As described in detail in Example 1, hereinabove.

Results

To gain insight into the potential intrinsic genes affected in the pig embryonic implants transplanted into hemophilic and non-hemophilic mice, inventors examined global gene expression using DNA microarray analysis. DNA microarray analysis was carried out for pig spleen implants in Factor VIII wild type and KO host mice at 8 weeks post transplant (data not shown). Differential expression of genes which are generally involved in cell growth, proliferation and apoptosis was demonstrated. However, differential expression of two gene families, the first associated with coagulation and endothelium and the second associated with patterning and differentiation, may be specifically relevant to the regulation of transplant organ size. For the complete data and discussion of the most potentially relevant genes identified by this analysis see Table 2, below.

TABLE 2
Selected gene families modulated in pig embryonic spleen
following implantation into Factor VIII KO SCID mice

| | Factor VIII KO-SCID versus | |
|---|---|---|
| | NOD-SCID regulation | |
| | (up or down regulation factor) * | |
| Coagulation and Endothelium | | |
| von Willebrand factor | 4 | Up regulated |
| Angiopoietin 1 | 3.8 | Up regulated |
| Tissue plasminogen activator | −3.2 | Down regulated |
| Tissue factor pathway inhibitor | −2.4 | Down regulated |
| Thrombospondin 4 precursor | −4.8 | Down regulated |
| Tissue factor | −7.4 | Down regulated |
| Differentiation and Pattering | | |
| Wnt-5b | 3.6 | Up regulated |
| LRP5 | 3.8 | Up regulated |
| LRP6 | 5.6 | Up regulated |
| Fibroblast growth factor receptor 1 | 3.4 | Up regulated |
| Fibroblast growth factor 18 | 2 | Up regulated |
| Epidermal growth factor | 4 | Up regulated |
| BMP-4c | 2.6 | Up regulated |
| Transforming growth factor-beta-1 | −2.4 | Down regulated |
| TGF-beta-activated kinase | −9.8 | Down regulated |
| TERT | 3.8 | Up regulated |
| Dickkopf related protein-3 | −2.6 | Down regulated |

* Regulation factor was calculated according to Affimetrix guidelines.

Several intrinsic pathways in the pig implants were affected by the absence of mouse Factor VIII (see Tables 1 and 2). For example, Wnt pathway components, such as Wnt5b, LRP5 and LRP6, were expressed at higher levels in spleens grown in Factor VIII KO SCID mice. The Wnt pathway was previously suggested to be an important regulator of organ size, for example Suksaweang et al. demonstrated that overexpression of active beta-catenin/Wnt, in an embryonic chicken model, lead to an enlarged liver with an expanded hepatocyte precursor cell population [Suksaweang, S. et al., Dev Biol (2004) 266, 109-22]. BMP4, which was shown to induce expression of numerous genes involved in Wnt signaling [Nishanian, T. G. et al., Cancer Biol Ther (2004) 3, 667-75], was also up regulated in Factor VIII KO SCID mice.

Fibroblast growth factor (FGF) signaling mediates cell-to-cell communication in development and organ homeostasis. A role for FGFR-1 and for FGF-18 in organ growth regulation has been previously demonstrated [Huang, X. et al., Cancer Res (2006) 66, 1481-90; Hu, M. C. et al., Mol Cell Biol (1998) 18, 6063-74] and was shown herein to be upregulated in Factor VIII KO SCID mice. Likewise, epidermal growth factor (EGF) was previously implicated in the control of visceral organ growth [Vinter-Jensen, L. et al., Growth Horm IGF Res (1998) 8, 411-9; Parker, J., Curr Biol (2006) 16, 2058-65] and was also demonstrated herein to be upregulated in Factor VIII KO SCID mice.

Transforming growth factor beta (TGF-beta), known to act as a negative autocrine growth factor, was down regulated in the Factor VIII KO SCID mice. Similarly, TGF-beta activated kinase 1 (TAK1) was down-regulated in Factor VIII KO SCID mice. Thus, TGF-beta may contribute to the increased organ size observed in hemophilic mice. TGF-beta and TAK1 were previously shown to repress the expression of the telomerase catalytic subunit (TERT) [Fujiki, T. et al., Oncogene (2007)]. The down regulation of both TGF-beta and TAK1 in the factor VIII KO SCID mice was therefore expected to result in increased expression of TERT, as was indeed shown (data not shown).

Dickkopf related protein-3 (Dkk3), whose expression was down-regulated in pig embryonic transplants grown in Factor VIII KO hosts, was previously shown to modulate both FGF signaling and TGF-beta signaling [Pinho, S, and Niehrs, C., Differentiation (2007)].

Clearly, interactions between members of the growth pathways discussed above may result in functional regulatory networks. For example, beta-catenin transgenic mice show an in vivo hepatotrophic effect secondary to increased basal hepatocyte proliferation [Tan, X. et al., Gastroenterology (2005) 129, 285-302]. Epidermal growth factor receptor seems to be a direct target of the Wnt/beta-Catenin pathway, and epidermal growth factor receptor activation might contribute to some of the mitogenic effects of increased beta-catenin in liver [Tan, X. et al., supra].

It was previously shown that alterations in expression of genes involved in coagulation, thrombosis and angiogenesis, such as Angiopoietin 1, may contribute to organ size determination [Metheny-Barlow, L. J. and Li, L. Y., Cell Res (2003) 13, 309-17]. As was demonstrated herein (see Table 2), the coagulation and endothelium factors von Willebrand factor and Angiopoietin 1 were upregulated in pig embryonic spleens following implantation in Factor VIII KO SCID mice.

In conclusion, inventors have shown in two different settings, namely transplantation of embryonic precursor tissues as well as in G-CSF induced splenomegaly, that Factor VIII, whose only known function is in blood coagulation, also exhibits a novel role in organ size control via regulation of genes.

### Example 4

### The Role of Coagulants Other than Factor VIII in Organ Size Control

Materials and Experimental Procedures

Animals

As described in detail in Example 2, hereinabove.

G-CSF Treatment

As described in detail in Example 2, hereinabove.

Clexane Treatment

Enoxaparine (Clexane 20 mg/0.2 ml, Rhone-poulenc, France) was used at a dosage of 200 μg/mouse (dissolved in PBS) and 0.2 ml of the final solution was injected subcutaneously into each mouse once a day.

Dabigatran Treatment

Dabigatran etexilate (Boehringer Ingelheim Pharma KG, Biberach, Germany) was administrated orally at dosage of 30 mg/kg. Final volume of 0.3 ml dissolved in DDW was administrated daily.

PAR1 and PAR4 Antagonists

The palmitoylated peptides: pal-RCLSSSAVANRS (PAR1 antagonist, SEQ ID NO: 15) and pal-SGRRYGHALR (PAR4 antagonist, SEQ ID NO: 16) were prepared by solid-phase peptide synthesis using in situ neutralization/HBTU by Hadar Biotec, Israel.

The mice were treated by vehicle control, or with PAR1 antagonist or PAR4 antagonist at 0.5 mg/kg, intraperitonealy on daily basis.

Transplantation Procedure

As described in detail in Example 1, hereinabove.

ELISA Measurements of Pig Albumin

As described in detail in Example 1, hereinabove.

Statistical Analysis

As described in detail in Example 1, hereinabove.

Results

Inhibition of Factor Xa and Thrombin by Clexane Enhances Organ Size

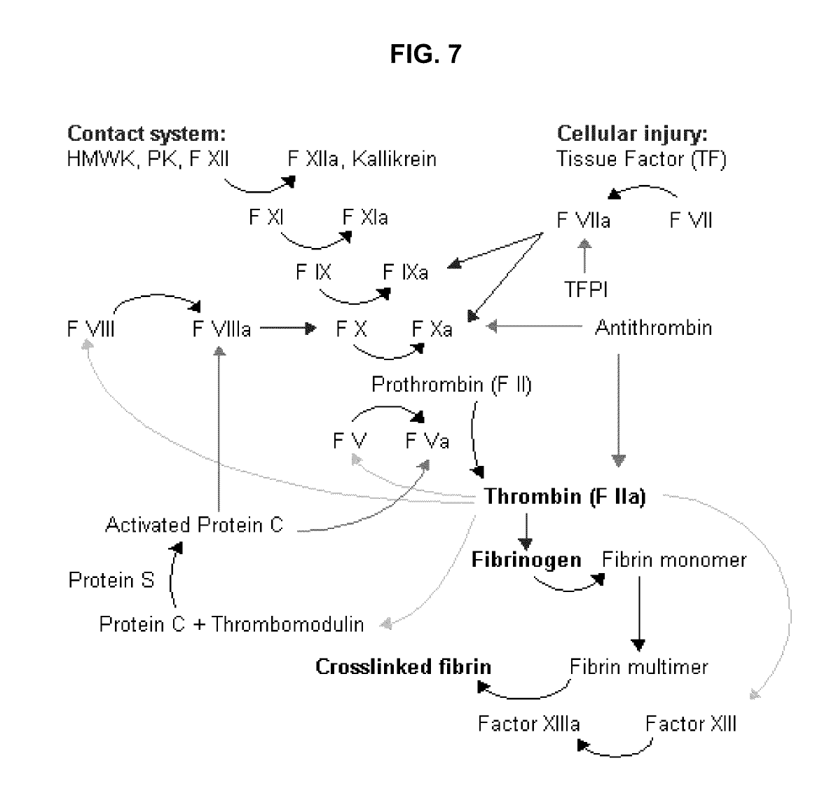

As described hereinabove, the present findings indicated a role for Factor VIII in organ size control. This intriguing role can either be mediated directly by Factor VIII and/or through one of the other coagulants activated along the cascade triggered by Factor VIII. As illustrated in FIG. 7, Factor Xa is activated by Factor VIII and, in turn, Thrombin is activated by Factor Xa. Thus, both factors could potentially mediate the observed enhancement of organ size.

One approach to blocking Factor Xa and, to a lesser degree, Thrombin is by the anti-coagulant Clexane, a low molecular weight heparin derivative. Clexane binds to and accelerates the activity of anti-thrombin III and thereby preferentially potentiates the inhibition of Factors Xa and IIa (thrombin) (see FIG. 8A).

As can be seen in FIG. 8B, Clexane administration exhibited a marked enhancement of the G-CSF induced splenomegaly. Indeed, the contribution of Clexane treatment to splenomegaly was as effective as that mediated by the Factor VIII KO mutation. Thus, following G-CSF induction, the total average weight of the spleens was 266±91 mg in C57BL/6 mice treated with Clexane compared to 261±154 mg in the Factor VIII KO C57BL/6 mice (data not shown).

In parallel to G-CSF experiments, pig liver transplantation model with and without Clexane administration was evaluated. As depicted in FIG. 8C, implantation of pig embryonic liver affords a rapid assay as growth can be monitored by the appearance of pig albumin (detectable by specific ELISA) in the mouse serum as early as 7 days post transplant. Importantly, Clexane administration in non-hemophilic recipients induced marked enhancements of pig albumin blood levels on days 7 and 21 post transplant compared to control recipients not receiving Clexane.

G-CSF Splenomegaly is Enhanced Upon Specific Inhibition of Thrombin by Dabigatran

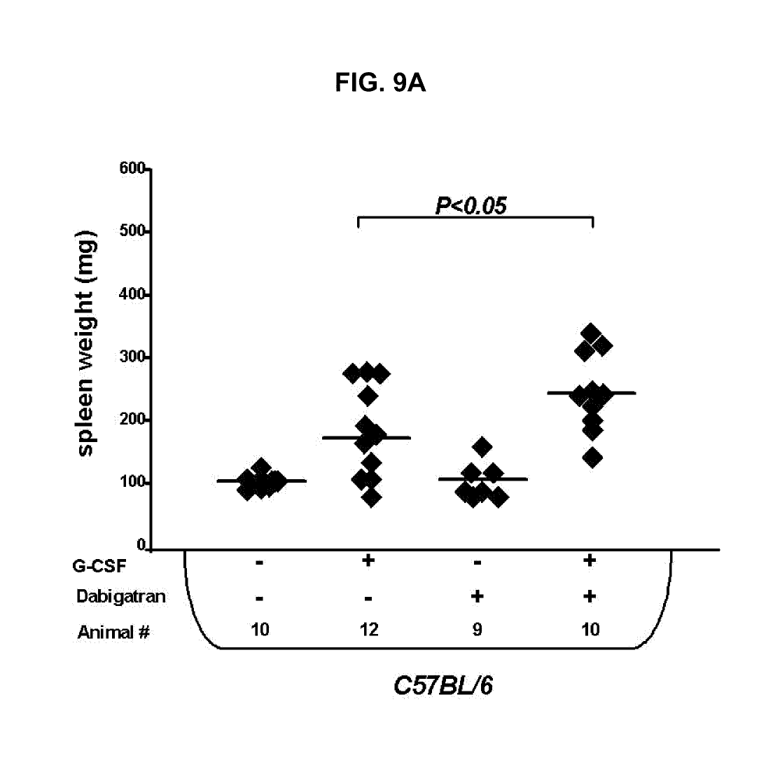

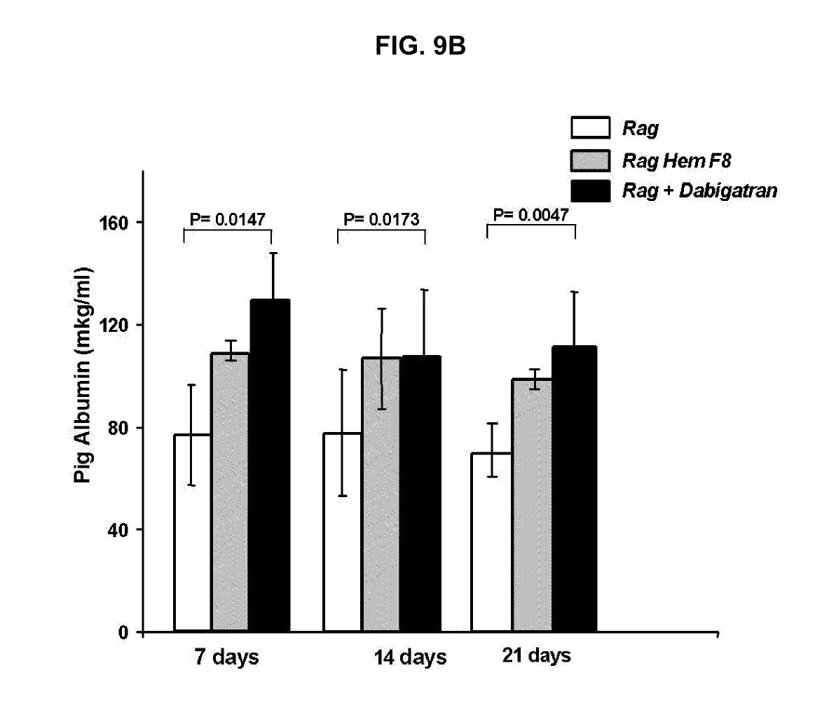

Considering that Clexane could potentially block not only factor Xa but to some extent also Thrombin, further analysis was performed using a more specific inhibitor of Thrombin, namely, Dabigatran. As can be seen in FIGS. 9A-B, Dabigatran administration led to marked enhancement of the G-CSF induced splenomegaly, similar to that exhibited by Clexane, and to an enhancement of embryonic pig liver growth. These results mark thrombin as a potential direct player in the enhanced splenomegaly phenotype (FIG. 9A) or in the enhancement of pig embryonic liver implants (FIG. 9B). Thus, thrombin and anti-thrombin may be important candidates for further manipulation of organ size.

Enhanced G-CSF Splenomegaly Upon Specific Inhibition of Thrombin by PAR1 and PAR4 Antagonists

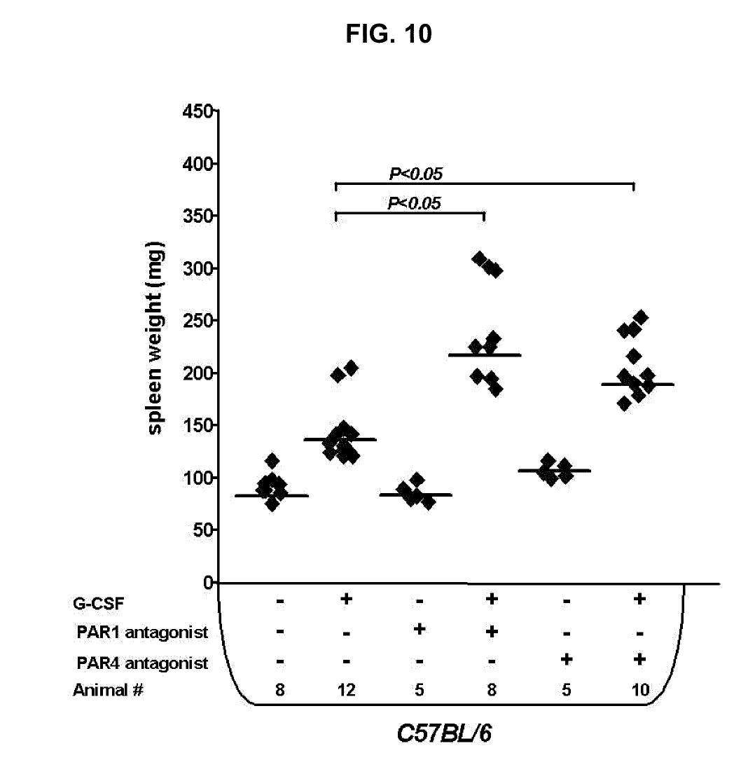

Since thrombin could operate through Protease-Activated Receptor (PAR) signaling and, especially, through a fine balance between the PAR1 and PAR4 receptors, inventors next evaluated the role of PAR signaling. To this end, inventors tested PAR1 and PAR4 antagonists (as explained in detail in the materials and experimental procedures section, hereinabove). As can be seen in FIG. 10, daily treatment with the PAR1 or PAR4 antagonist significantly enhanced G-CSF induced splenomegaly (p<0.05).

### Discussion

The models described above making use of heterologous embryonic transplantation provided an additional tool in the study of growth control, in that it enabled investigation of the cross-talk between extrinsic host factors and intrinsic mechanisms relevant to size control in the implant. In particular, this assay enabled pinpointing the role of different extrinsic genes by using mutated or KO host mice. Thus, inventors demonstrated, for the first time, that host Factor VIII plays a critical role as an extrinsic factor in controlling the final size of pig-derived organs.

The lack of any effect on mouse embryonic transplants, regardless of whether the host or the donor was derived from a hemophilic or non-hemophilic mouse, suggested that the role of Factor VIII is likely limited to a growth checkpoint that only operates upon implantation of tissue from a larger animal, such as the pig. Thus, assuming that mouse and pig embryonic implants are endowed with stem cell pools of different size prior to transplantation, they are likely to attain different organ sizes upon completion of growth and differentiation in the mouse recipients. Consequently, in hemophilic mice lacking the potential inhibitory activity (i.e. overgrowth checkpoint) mediated by Factor VIII, the size of the pig implants is likely to be larger, while mouse implants growing to their normal expected size will not exhibit excessive growth, and therefore will not be subject to Factor VIII control.

It could be argued that the role of Factor VIII might be limited to the transplantation setting and thus might not have a true physiological role in the intact animal. However, the results obtained in the context of G-CSF induced splenomegaly, extended these findings to yet another system in which a stimulus for overgrowth is also limited by factors of the coagulation cascade.

Further interrogation of other factors downstream of Factor VIII in the coagulation cascade revealed that thrombin is probably the Factor VIII downstream factor that actually mediates regulation of oversize control.

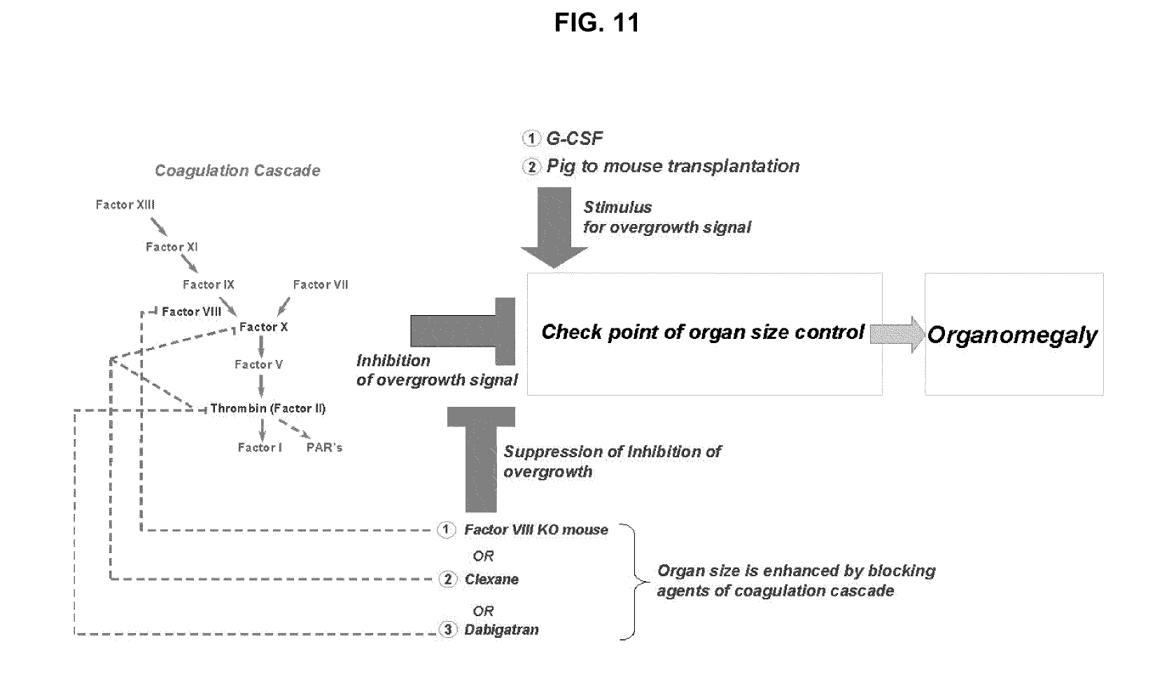

Thus, as outlined schematically in FIG. 11, interference with steady state levels of factors of the coagulation cascade, as occurs in Factor VIII KO mice or upon blockade of Factor Xa or thrombin, also overcomes this checkpoint, thereby leading to oversized organs or to the enhancement of G-CSF induced splenomegaly. The suggestion that thrombin is a likely candidate for the actual inhibitory activity leading to oversize control, is supported by its terminal position in the hemostatic system, acting downstream of Factor VIII and Factor Xa. This hypothesis was further supported by the demonstration that blockade of thrombin receptors PAR1 or PAR4, similarly to thrombin blockade by Dabigatran, led to enhancement of G-CSF induced splenomegaly.

In conclusion, the present data suggest a novel role for factors of the coagulation cascade in organ size control. In particular, a role for thrombin was pinpointed. This surprising finding may not only offer new means to enhance or reduce the final size of implanted xenogeneic embryonic tissues, but also adds a novel insight into the mysterious question of organ size control in mammals.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of modulating transplant organ size in a subject in need thereof, the method comprising:
(a) administering to the subject an agent capable of modulating an activity or expression of a coagulation factor or an effector thereof; and
(b) transplanting the organ into the subject;
thereby modulating the transplant organ size in the subject.

2-5. (canceled)

6. An article of manufacture comprising a packaging material packaging an immunosuppressing agent and an agent capable of modulating an activity or expression of a coagulation factor or an effector thereof.

7. The method of claim 1, wherein said modulating transplant organ size comprises enhancing the transplant organ size.

8. The method of claim 7, wherein said agent is capable of down-regulating said activity or expression of said coagulation factor or an effector thereof.

9. The method of claim 8, wherein said coagulation factor or an effector thereof is selected from the group consisting of Factor VIII, Factor X, Factor Xa, Prothrombin, Thrombin, Factor XIII, Factor XIIIa and PAR.

10. The method of claim 7, wherein said agent is capable of down-regulating an activity or expression of a coagulation factor selected from the group consisting of Factor VIII, Factor Xa and thrombin.

11. (canceled)

12. The method of claim 10, wherein when said factor is Factor Xa, said agent is Clexane.

13. (canceled)

14. The method of claim 10, wherein when said factor is thrombin, said agent is selected from the group consisting of Clexane and Dabigatran.

15. The method of claim 7, wherein said agent is capable of up-regulating an activity or expression of antithrombin in the subject.

16. The method of claim 7, wherein said agent is capable of down-regulating an activity or expression of PAR1 in the subject.

17. The method of claim 16, wherein said agent is as set forth in SEQ ID NO: 15.

18. The method of claim 7, wherein said agent is capable of down-regulating an activity or expression of PAR4 in the subject.

19. The method of claim 18, wherein said agent is as set forth in SEQ ID NO: 16.

20. The method of claim 12, wherein said agent further comprises G-CSF.

21. (canceled)

22. The method of claim 1, wherein said modulating transplant organ size comprises decreasing the organ size.

23. The method of claim 22, wherein said agent is capable of up-regulating said activity or expression of said coagulation factor or an effector thereof.

24-40. (canceled)

41. The method of claim 1, wherein said subject is a human being.

42-44. (canceled)

45. The method of claim 1, wherein said modulating an activity or expression of a coagulation factor or an effector thereof is effected prior to, concomitantly with or following transplantation.

46. The method of claim 1, further comprising conditioning the subject prior to transplanting so as to prevent organ rejection.


SEQUENCE LISTING (16 sequences)

SEQ ID NO: 1 - RNA, 20 nt, Artificial sequence
  Phosphorothioate backbone antisense oligonucleotide targeting Factor VIII mRNA
guccacuugcagccacucuu

SEQ ID NO: 2 - RNA, 19 nt, Artificial sequence
  Phosphorothioate backbone antisense oligonucleotide targeting Factor VIII mRNA
guccacuugcagccacucu

SEQ ID NO: 3 - RNA, 21 nt, Artificial sequence
  Phosphorothioate backbone antisense oligonucleotide targeting Factor VIII mRNA
guccacuugcagccacucuuu

SEQ ID NO: 4 - RNA, 19 nt, Artificial sequence
  Phosphorothioate backbone antisense oligonucleotide targeting Factor VIII mRNA
gcuuuacucuccauuccca

SEQ ID NO: 5 - RNA, 20 nt, Artificial sequence
  Phosphorothioate backbone antisense oligonucleotide targeting Factor VIII mRNA
ugcuuuacucuccauuccca

SEQ ID NO: 6 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - DNA, 9048 bp, Homo sapiens
gcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccatttggtcttaattctgaccaataaaaaaataagtcaggaggatgcaattgttgaaagctttgaaataaaataacaatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgaccccttcactatgactacacagaatctcctgatagtaaagggggctggaggcaaggataagttatagagcagttggaggaagcatccaaagattgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacatacaaacaaaaaaaaaaaaaaa

SEQ ID NO: 8 - protein, 216 aa, Homo sapiens
MRIQDPGKVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 9 - DNA, 1560 bp, Homo sapiens
gactttgctccagcagcctgtcccagtgaggacagggacacagtactcggccacaccatggggcgcccactgcacctcgtcctgctcagtgcctccctggctggcctcctgctgctcggggaaagtctgttcatccgcagggagcaggccaacaacatcctggcgagggtcacgagggccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaagtgagatcccactcaaaaaaaaaaaaaaaaaaaaaaaaaa

SEQ ID NO: 10 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 11 - DNA, 1997 bp, Homo sapiens
aattcctcagtgacccaggagctgacacactatggcgcacgtccgaggcttgcagctgcctggctgcctggccctggctgccctgtgtagccttgtgcacagccagcatgtgttcctggctcctcagcaagcacggtcgctgctccagcgggtccggcgagccaacaccttcttggaggaggtgcgcaagggcaacctagagcgagagtgcgtggaggagacgtgcagctacgaggaggccttcgaggctctggagtcctccacggctacggatgtgttctgggccaagtacacagcttgtgagacagcgaggacgcctcgagataagcttgctgcatgtctggaaggtaactgtgctgagggtctgggtacgaactaccgagggcatgtgaacatcacccggtcaggcattgagtgccagctatggaggagtcgctacccacataagcctgaaatcaactccactacccatcctggggccgacctacaggagaatttctgccgcaaccccgacagcagcaccacgggaccctggtgctacactacagaccccaccgtgaggaggcaggaatgcagcatccctgtctgtggccaggatcaagtcactgtagcgatgactccacgctccgaaggctccagtgtgaatctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgtgtggtgctatgtggccgggaagcctggcgactttgggtactgcgacctcaactattgtgaggaggccgtggaggaggagacaggagatgggctggatgaggactcagacagggccatcgaagggcgtaccgccaccagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcacaaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtaggggccactcatattctgggctcctggaaccaatcccgtgaaagaattatttttgtgtttctaaaactatggttcccaataaaagtgactctcagcgg

SEQ ID NO: 12 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 13 - protein, 5 aa, Artificial sequence
  PAR1 agonist peptide
TFLLR

SEQ ID NO: 14 - protein, 6 aa, Artificial sequence
  PAR4 agonist peptide
AYPGKF

SEQ ID NO: 15 - protein, 12 aa, Artificial sequence
  PAR1 antagonist peptide
RCLSSSAVANRS

SEQ ID NO: 16 - protein, 10 aa, Artificial sequence
  PAR4 antagonist peptide
SGRRYGHALR